(19) United States
(12) Patent Application Publication
(10) Pub. No.: US 2014/0154224 A1
(43) Pub. Date: Jun. 5, 2014

(54) Method of Pest Control

(71) Applicants: Beijing Dabeinong Technology Group Co., Ltd., Beijing (CN); Beijing Green Agrosino Plant Protection Technology Co., Ltd., Beijing (CN); Beijing Dabeinong Technology Group Co., Ltd., Biotech Center, Beijing (CN)

(72) Inventors: Shengbing LI, Beijing (CN); Yuejing KANG, Beijing (CN); Peng CHENG, Beijing (CN); Ruiqi NIU, Beijing (CN); Jing LIU, Beijing (CN); Xiaoqian SONG, Beijing (CN); Li LIU, Beijing (CN); Jinfeng ZHANG, Beijing (CN); Kangle TIAN, Beijing (CN); Yanxiao LIU, Beijing (CN)

(73) Assignees: Beijing Dabeinong Technology Group Co., Ltd., Beijing (CN); Beijing Green Agrosino Plant Protection Technology Co., Ltd., Beijing (CN); Beijing Dabeinong Technology Group Co., Ltd., Biotech Center, Beijing (CN)

(21) Appl. No.: 14/095,050

(22) Filed: Dec. 3, 2013

(30) Foreign Application Priority Data
Dec. 3, 2012 (CN) 201210511214.6

(51) Int. Cl. C12N 15/82; A01N 37/46

(52) U.S. Cl. CPC C12N 15/8286; A01N 37/46
USPC 424/93.21; 514/4.5; 800/302

(57) ABSTRACT

The present invention provides a method for controlling Conogethes punctiferalis, which comprises contacting Conogethes punctiferalis with Cry1A protein. The present invention achieves the control of Conogethes punctiferalis by enabling plants to produce Cry1A protein in vivo, which is lethal to Conogethes punctiferalis. In comparison with current agricultural and chemical control methods, the method of the present invention can control Conogethes punctiferalis throughout the growth period of the plants and provide the plants with a full protection. Additionally, the method is stable, complete, simple, convenient, economical, pollution-free and residue-free.

### FIELD OF THE INVENTION

The present invention relates to a method for pest control, specifically a method for preventing Conogethes punctiferalis from making damages to plants by Cry1A protein expressed therein.

### BACKGROUND

Conogethes punctiferalis belongs to Lepidoptera, Crambidae. As an omnivorous pest, it damages not only crops like maize and sorghum but also fruit trees such as peach, persimmon and chestnut trees. It is widely distributed in China, north from Heilongjiang and Inner Mongolia; south to Taiwan, Hainan, Guangdong, Guangxi and the southern edge of Yunnan; east from eastern territory of the former Soviet Union and northern territory of North Korea; west to Shanxi, Shaanxi and after west ramp to Ningxia, Gansu, turning to Sichuan, Yunnan and Tibet. When feeding on maize, it mainly eats ears and sometimes stems, causing damaged to 30%-80% of the plants. When feeding on sorghum, hatched larvae invade into immature grains of the sorghum, then seal holes with feces or food residues, where they eat grains one by one until instar 3. After instar 3, they spin silk to conjugate spikelets into tunneled web, in which they chew the grains so that nothing is left in serious cases. In addition, it can damage stems, similar to Ostrinia furnacalis.

As Maize and sorghum are important food crops in China, damages of them by Conogethes punctiferalis are causing huge food loss every year and even effects on the living conditions of local people. Currently, there are three commonly used methods to control Conogethes punctiferalis: agricultural control, chemical control and biological control methods.

The agricultural control method is an integrated and coordinated management of multiple factors of the entire ecosystem of farmland, which regulates crops, pests and environmental factors and establishes a farmland ecosystem conducive to crop growth but unfavorable to Conogethes punctiferalis. For example, treating overwintering hosts of Conogethes punctiferalis, picking up and destroying fallen fruits, removing fruits damaged by pests, reforming farming rules, planting plants resistant to Conogethes punctiferalis, and planting trapping fields are done, in order to reduce the harm of Conogethes punctiferalis. Since the agricultural control method must obey the requirements for crop layout and increasing production, it has limited application and cannot be used as an emergency measure when Conogethes punctiferalis outbreaks.

The chemical control method, also known as pesticide control, is to kill pests by using pesticides. As an important means for the comprehensive management of controlling, it is fast, convenient, simple and highly cost-effective. Particularly, it can be used as an essential emergency practice to control Conogethes punctiferalis before damages has done. Currently, the major measures of the chemical control include chemical trapping, liquid spray and the like. However, the chemical control has its limitations: its improper use can cause devastating consequences, such as poisoning crops, pest resistance, killing predators and polluting the environment so as to destroy farmland ecosystems; pesticide residues pose a threat to the safety of local human and livestock.

The biological control method uses certain beneficial organisms or biological metabolites to control pest populations, achieving the purpose of reducing or eliminating pests. It had many advantages including causing no harms to human and livestock, bringing little pollution to the environment, and long-term controlling of certain pests. However, its effects are often unstable and it requires same amount of investments regardless the severity of damages caused by Conogethes punctiferalis.

To overcome the limitations of the agricultural control, chemical control and biological control methods, researchers have found that the insertion of genes coding for pesticidal proteins into plant genome can produce pest-resistant plants. Pesticidal protein Cry1A, among a large group of pesticidal proteins, is an insoluble parasporal crystal protein produced by a subspecies of Bacillus thuringiensis (Bacillus thuringiensis subsp.kurstaki, B.t.k).

Cry1A protein, if ingested by pests, is dissolved in the alkaline environment of the pests' midgut and releases protoxin, a precursor to a toxin. Further, alkaline protease digests the protoxin at its N- and C-terminus and produces an active fragment, which would subsequently bind to a membrane receptor of epithelial cells of the pests' midgut and insert itself into the intestinal membrane, resulting in cell membrane perforation, disequilibrating the pH homeostasis and osmotic pressure across the cell membrane, disturbing the digestion of the pest, and eventually leading to the death of the pests.

It has been confirmed that Cry1Ab transgenic plants can resist Lepidoptera pests such as maize borer, cotton bollworm and stem borer. However, thus far there are no reports on controlling Conogethes punctiferalis by generating transgenic plants producing the Cry1A protein.

### SUMMARY OF THE INVENTION

The purpose of the present invention is to provide a pest control method for the first time using a transgenic plant expressing Cry1A protein to control damage caused by Conogethes punctiferalis. Said method effectively overcomes technical limitations of the agricultural control and chemical control methods.

To achieve the purpose as mentioned above, the present invention provides a method for controlling Conogethes punctiferalis, said method comprises contacting Conogethes punctiferalis with the Cry1A protein.

Additionally, the present invention provides a transgenic plant expressing the Cry1A protein and its reproductive material, such as seeds, seedlings and the like.

Preferably, the Cry1A protein is Cry1Ab protein or Cry1Ah protein.

Further, the Cry1Ab protein is present in a cell expressing the Cry1Ab protein of a plant, and it is contacted with Conogethes punctiferalis by ingestion of the cell.

Further, the Cry1Ab protein is present in a transgenic plant expressing the Cry1Ab protein, and Conogethes punctiferalis contacts with the Cry1Ab protein by ingestion of a tissue of the transgenic plant. As a consequence, the growth of Conogethes punctiferalis is inhibited, leading to the death of Conogethes punctiferalis eventually, and then damage of Conogethes punctiferalis to the plant is controlled.

Further, the Cry1Ah protein is present in a cell expressing the Cry1Ah of a plant, and it is contacted with Conogethes punctiferalis by ingestion of the cell.

Further, the Cry1Ah protein is present in a transgenic plant expressing the Cry1Ah protein, and it is contacted with Conogethes punctiferalis by ingestion of a tissue of the transgenic plant. As a consequence, the growth of Conogethes punctiferalis is inhibited, leading to the death of Conogethes punctiferalis eventually, and then the damage of Conogethes punctiferalis to the plant is controlled.

The transgenic plant can be in any growth period.

The tissue of the transgenic plant can be roots, leaves, stems, tassels, ears, anthers or filaments.

The control of the damage of Conogethes punctiferalis to the plant does not depend on planting location.

The control of the damage of Conogethes punctiferalis to the plant does not depend on planting time.

The plant can be derived from maize, sorghum, millet, sunflower, castor, ginger, cotton, peach, persimmon, walnut, chestnut, fig or pine.

Prior to the step of contacting is to plant a transgenic seedling that contains a polynucleotide encoding the Cry1A protein.

Preferably, the amino acid sequence of the Cry1A protein comprises an amino acid sequence of SEQ ID NO:1, SEQ ID NO:2 or SEQ ID NO:3. The nucleotide sequence encoding the Cry1A protein comprises a nucleotide sequence of SEQ ID NO: 4, SEQ ID NO:5, SEQ ID NO:6 or SEQ ID NO:7.

Based on the technical solutions mentioned above, the plant can further contain at least one of a second nucleotide, which is different from the Cry1A protein.

Further, the second nucleotide can encode a Cry-like pesticidal protein, a Vip-like pesticidal protein, a protease inhibitor, lectin, α-amylase or peroxidase.

Preferably, the second nucleotide can encode Cry1Ie protein, Cry1Fa protein, Vip3A protein or Cry1Ba protein.

Further, the second nucleotide comprises a nucleotide sequence of SEQ ID NO:8 or SEQ ID NO:9.

Optionally, the second nucleotide is dsRNA, which inhibits an important gene of a target pest.

In the present invention, the expression of Cry1A protein in one transgenic plant can be accompanied by the expression of one or more Cry-like pesticidal proteins and/or Vip-like pesticidal proteins. Such co-expression of more than one pesticidal toxins in the same transgenic plant can be achieved by genetic engineering to make the plant containing and expressing genes of interest. In addition, one plant (the first parent) expresses Cry1A protein by genetic engineering, while another plant (the second parent) expresses Cry-like pesticidal protein and/or Vip-like pesticidal protein by genetic engineering. Crossing the first parent with the second parent obtains progeny plants expressing all of the genes introduced into the first parent and the second parent.

RNA interference (RNAi) is referred as a phenomenon of high specific and efficient degradation of homologous mRNA induced by double-stranded RNA (dsRNA), which is highly conserved during evolution. Therefore, the present invention can use RNAi to specifically knockout or close the expression of genes of target pests.

Although both Conogethes punctiferalis and Ostrinia nubilalis belong to the family Crambidae of the order Lepidoptera and are omnivorous pests, they are clearly two distinct species in biology. Conogethes punctiferalis and Ostrinia nubilalis have at least the following differences:

1. Different feeding habits. In addition to Gramineous plants such as maize, Conogethes punctiferalis also feed on fruit trees including peaches, pomegranates, chestnut and persimmon. Whereas Ostrinia nubilalis mainly feed on Gramineous plants, mostly maize and sorghum.
2. Different geographical habitations. Conogethes punctiferalis inhibits not only throughout the whole territory of China but also other places such as Japan, the Korean Peninsula, UK and Australia. Ostrinia nubilalis consists of Asian corn borer and European corn borer. The Asian corn borer inhabits mainly at maize and sorghum production areas in the east and southwest part of China, whereas the European corn borer is mostly found in Xinjiang (China), Europe, North America and Asia Minor. In regard to geographical habitations, Conogethes punctiferalis is much widely distributed than both Asian corn borer and European corn borer.
3. Different infestation habits. When feeding on sorghum, firstly hatched larvae of Conogethes punctiferalis enter into immature grains of the sorghum, then seal holes with their feces or food residues, where they eat grains one by one until instar 3. After instar 3, they spin silk to conjugate spikelets into tunneled webs, in which they chew the grains until nothing left in serious cases. In addition, it can damage stems. When damaging maize, they mainly target ears. After eating into immature grains, they produce sticky feces to seal the wormhole then damage inside. Meanwhile they can also feed on stems, leaves and grains. When infestation, Conogethes punctiferalis often produce sticky feces, which increase the occurrence of molds, especially Aspergillus flavus, thus affect the feed processing. The larvae of Ostrinia nubilalis will gather together after hatch, then crawl over and damage the immature parts of the plants. Newly hatched larvae are able to transfer themselves to the neighboring plants: they spin silk and hang themselves from the damaged plant and the wind will blow them to their next targets. The larvae go through 5 instars: before instar 3, they mainly feed on young heart leaves, tassels, husks and filaments, leaving lots little holes in horizontal setting, which only visible by unfolding the heart leaves; after instar 4, they eat in the stems. They can cause functional damages to every part of maize plants above the ground: the leaves reduce their photosynthetic efficiency after damaged; the broken tassels lose their ability of pollination; the damaged husks and filaments lead to blasted corns; the bitten stems, pedicels and cob have tunnels formed inside, impeding the transportation of nutrition and water to the plants, leading to an increased stalk fracture rate and a decreased corn yield rate. Spring, summer and autumn maize in all regions can be damaged in various degrees, wherein autumn maize suffers the most.
4. Different morphological features.
1) Different morphology of eggs: Conogethes punctiferalis's eggs are 0.6-0.7 mm in length, and have a rough surface with full of tinny round speckles or reticular patterns. The eggs are orange-red before hatch and are individually distributed on a rough surface. While Ostrinia nubilalis' eggs are in flat oval shape, with dozens of black fish-scale like spots irregularly aligned on top of the eggs before hatch.
2) Different morphology of larvae: the bodies of Conogethes punctiferalis's larvae have various color from light gray to dark red: the abdomen is mostly light green, the head is fuscous, the back is dark red, the abdomen is light green, and the prothorax and pygidium are dark brown. Each body segment of the larvae has clear pinaculums with color from taupe to black brown. Each of the first to eighth abdominal segments has eight pinaculums, and they are aligned in two rows: the front row comprises six bigger ones while the back row has two smaller ones. After instar 3, two dark brown gonads appear on the fifth abdominal segment of male larvae. By contrast, the larvae of Ostrinia nubilalis have white-yellow to fuscous backs, dark brown heads and prothoraxs, clear dorsal lines, vague dark brown sub dorsal lines at both sides, and 2 lines of verrucae at the first to eighth abdominal segments (4 in front line and 2 in bottom line).
3) Different morphology of pupae: the pupae of Conogethes punctiferalis are pale yellowish green at early stage then tuning black brown. The head and the back of first to eighth abdominal segments are densely covered by tinny protrusions, and the anterior border of fifth to seventh abdominal segments has one raised line formed by small denticulate protrusions. At the end of the abdomen, there are six slender and curly hooked spines. While the pupae of Ostrinia nubilalis are tawny with an intense pattern of horizontal ventrodorsal wrinkles, and 5-8 spines at the bottom of abdomen.
4) Different morphology of imago: adult Conogethes punctiferalis are yellow to orange with a lot of black dots at the thorax, abdomen and wings. Two pilose sides of prothorax both have one black dot. The end of the ninth abdominal segment of male moth is clearly black, fairly truncated and has black floccus. The end of the abdomen of female moth is conical, and in the last segment, there are few black flakes only at the end of the back. By contrast, adult Ostrinia nubilalis has taupe hindwings, and tawny forewings with two brown wavy horizontal strips, between which there are two short tawny strips. Comparing to the male, the female have light-color wings; less obvious endo-transverse lines, external transverse line and speckles, and their forewings are yellow.
5. Different growth habit. The number of generations in a year varies geographically for Conogethes punctiferalis grow: 1-2 generations in Liaoning Province, while 3 generations in Hebei, Shandong or Shaanxi Province, 4 generations in Henan Province, and 4-5 generations. in Yangtze River valley All of them live through the winter via cocooning in the stubbles of maize, sunflower, castor oil plant, etc. by fully matured larvae. In Henan Province, the larvae of the first generation damage peach from late May to late June, the larvae of generations 2 and 3 damage both peach and sorghum while the larvae of generation 4 damage sorghum and sunflower in summer seedings. The larvae of generation 4 live through the winter. In the next year, survived winter larvae pupate in the beginning of April and enter the peak period of pupation in the late April. The larvae enter eclosion from the end of April to the late May and imagoes of the over-wintering generation lay eggs on peach. The larvae of the first generation pupate from the middle of June to the late June and the imagoes of the first generation start to appear on the late June and then enter the peak period of eclosion in early July. This is followed by the peak period of eclosion of the second generation when spring seeding sorghum become earing and flowering. The damage of the larvae of the second generation is the most severe in the middle of July. The peak period of eclosion of the second generation is in the early and middle of August, when spring sorghum is approaching maturity and late-sowing spring sorghum and early-sowing summer sorghum are earing and flowering. The imagoes are laying eggs mainly on the sorghum. The eggs of the third generation are hatched on the end of July or early August. The damage of the larvae of the third generation is the most severe in the middle and late of August. The imagoes of the third generation appear in the end of August and become prosperous in the early and middle of September, when sorghum and peach fruits have been harvested. The imagoes lay eggs on late summer sorghum and late-maturing sunflower. The damage of the larvae of the fourth generation is from the middle of September to the middle of October. The larvae of the fourth generation live through the winter when temperature drops in the middle and late October. In Henan Province, the eggs stage of the first generation is 8 days, while that of the second generation is 4.5 days, third is 4.2 days and the over-wintering generation is 6 days. The larval stage of the first generation is 19.8 days, while that of the second generation is 13.7 days, third is 13.2 day and the over-wintering generation is 208 days. Larvae have 5 instars. The pupal stage of the first generation is 8.8 days, while that of the second generation is 8.3 days, third is 8.7 days and the over-wintering generation is 19.4 days. The imago life of the first generation is 7.3 days, while that of the second generation is 7.2 days, third is 7.6 days and the over-wintering generation is 10.7 days. After eclosion, the imagoes hide in the sorghum field and only lay eggs through extra-nutrition, and they lay eggs on the earing and flowering sorghum. The eggs are single birth and each female imago lays 169 eggs with 3-5 eggs on one ear. In Yibin of Sichuan Province, the imagoes lay eggs on cars and tassels, commissure of leaf sheath or front and back of auricular from tasseling stage to waxen maturity stage of autumn maize, and amounts of the eggs are up to 1729 every one hundred of maize. After maturation the larvae live through the winter in ear or leaf axil, blade sheath, withered leaf and staw of sorghum, maize and sunflower. The damage is severe in the rainy year. In recent years there have been abundant rainfalls in Northern and Northeastern China because of the affect of global greenhouse effect, which makes that Conogethes punctiferalis become primary pest to maize in North China. But because they eat miscellaneous foods and often transfer between different hosts and are most fond of entering and boring through fruits and ears and stems, common pesticide spray is hard to control them. By contrast, the number of generations of Ostrinia nubilalis in China is significantly different according to the latitude: one generation if above the 45° N, two generations if between 45° N and 40° N, three generations if between 40° N and 30° N, four generations if between 30° N and 25° N, and five to six generations if between 25° N and 20° N. The higher of altitude, the fewer generations it can develop. In Sichuan province, a low altitude area with high temperature, it can develop two to four generations. The mature larvae normally enter inside the stems and cobs of maize, or straws of sorghum and sunflower till next year April to May when they are ready to pupate, which would take around 10 days. The adults have 5- to 10-day lifespan and are active during nights with great flight ability and phototaxis. The female prefer to lay their eggs on both sides of costae of corn leaves that flourishing 50 cm above the ground and each female can produce 350-700 eggs during its ovipositing period of 3-5 days. Ostrinia nubilalis is developing well under high temperature and humidity conditions. In hot winters, when there are also less parasitic enemies that favor their reproduction, Ostrinia nubilalis are causing greater damages. In contrast, Ostrinia nubilalis will cause less damage if they oviposite under drought condition, as corn leaves will curl up so that their eggs will drop and die easily.

Collectively, it is evident that Conogethes punctiferalis and Ostrinia nubilalis are two distinct species and cannot crossbreed.

In the present invention, the genome of plants, plant tissues or plant cells refers to any genetic material in the plants, plant tissues or cells, including nucleus, plastid and mitochondrial genome.

In the present invention, polynucleotides and/or nucleotides constitute a complete “gene”, and encode a protein or polypeptide in desired host cells. The skilled person in the art would readily recognize that the polynucleotides and/or nucleotides could be placed under the control of regulatory sequences of target hosts.

It would be well known for the skilled person in the art that DNA normally exists in a form known as double-stranded structure. In this arrangement, one strand is complementary with another strand, and vice versa. DNA generates other complementary strands during replication in plants, thus the present invention includes use of the polynucleotides exemplified in the sequence list and their complementary strands. “Coding strand” commonly used in the art refers to the strand binding to the antisense strand. In order to express proteins in vivo, one strand of DNA is typically transcribed into a complementary strand as mRNA, which is used as a template to be translated into protein. In fact, mRNA is transcribed from the “antisense” strand of DNA. “Sense” or “encoding” strand has a series of codons (one codon contains three nucleotides, which encodes a specific amino acid), and the strand can be used as an open reading frame (ORF) and be transcribed into a protein or peptide. The present invention also encompasses RNA and peptide nucleic acid (PNA), which have considerable functions as the exemplified DNA.

In the present invention, the nucleic acid molecules or fragments thereof hybridize to Cry1A gene of the present invention under stringent conditions. Any conventional nucleic acid hybridization or amplification method can be used to identify the presence of Cry1A gene of the present invention. The nucleic acid molecules or fragments thereof in certain cases can specifically hybridize to other nucleic acid molecules. In the present invention, if two nucleic acid molecules can form an antiparallel double-stranded nucleic acid structure, we can say that these two nucleic acid molecules can specifically hybridize to each other. If two nucleic acid molecules exhibit complete complementarity, one nucleic acid molecule is called the “complement” of the other nucleic acid molecule. In the present invention, when every nucleotide of one nucleic acid molecule is complementary to the corresponding nucleotide of another nucleic acid molecule, these two nucleic acid molecules are called to exhibit “complete complementarity”. If two nucleic acid molecules can hybridize to each other at an efficiently stable status, and bind to each other after annealing under at least conventional “low stringency” conditions, these two nucleic acid molecules are called “minimal complementarity”. Likewise, if two nucleic acid molecules can hybridize to each other at an efficiently stable status, and bind to each other after annealing under conventional “high stringency” conditions, these two nucleic acid molecules are called to have “complementarity”. Deviation from complete complementarity is acceptable as long as such deviation does not completely prevent the two molecules from forming a double-stranded structure. In order to ensure that a nucleic acid molecule can be used as a primer or probe, its sequence must have sufficient complementarity so that it can form a stable double-stranded structure in particular solvents and salt concentrations.

In the present invention, a substantially homologous sequence is a nucleic acid molecule, which, under highly stringent conditions, can specifically hybridize with the matched complementary strand of the other nucleic acid molecule. The stringent conditions suitable for DNA hybridization, e.g., processing with 6.0× sodium chloride/sodium citrate (SSC) at about 45° C. and then washing with 2.0×SSC at 50° C., would be well known to the skilled person in the art. For example, during the wash step the salt concentration can be selected from a low stringency condition of about 2.0×SSC to a highly stringent condition of about 0.2×SSC at 50° C. In addition, the temperature in the wash step can be selected from a low stringency condition of room temperature about 22° C. to a highly stringent condition of about 65° C. Both temperature and salt concentration can be changed, or one can remain intact while another one is changed. Preferably, the stringent conditions according to the invention are: specific hybridization with SEQ ID NO: 4, SEQ ID NO: 5, SEQ ID NO: 6 or SEQ ID NO: 7 in a 6×SSC, 0.5% SDS solution at 65° C., and then membrane washing with 2×SSC, 0.1% SDS and 1×SSC, 0.1% SDS once each.

Therefore, sequences having pest-resistant activity and capable of hybridizing with SEQ ID NO: 4, SEQ ID NO: 5, SEQ ID NO: 6 or SEQ ID NO: 7 under stringent conditions are encompassed in the present invention. These sequences are at least of about 40%-50% homology to the sequences of the present invention, about 60%, 65% or 70% homology, or even at least of about 75%, 80%, 85%, 90%, 91%, 92%, 93%, 94%, 95%, 96%, 97%, 98%, 99% or greater homology to the sequences of the present invention.

The genes and proteins described in the present invention include not only the specifically exemplified sequences, but also the portions and/fragments (including those having internal and/or terminal deletions in comparison with the full-length proteins), variants, mutants, substitutes (proteins with substituted amino acids), chimeric and fusion proteins thereof having the pesticidal activity of the exemplified proteins. The “mutants” or “variants” refer to nucleotide sequences encoding the same protein or equivalent protein with the pesticidal activity. The “equivalent protein” refers to a protein presenting the same or substantially the same biological activity of resistance to Conogethes punctiferalis as the claimed proteins.

The “fragment” or “truncation” of the DNA or protein sequences described in the present invention refers to a part or an artificially modified form (such as sequences suitable for plant expression) of the original DNA or protein sequences (nucleotides or amino acids). The length of the above sequences can be variable, but it should be sufficient to ensure the protein (encoded) as an pest toxin.

Genes can be easily modified as gene variants by standard techniques. For example, the technology of point mutation is well known in the art. Another example based on U.S. Pat. No. 5,605,793 describes a method that DNA can be reassembled to generate other molecular diversity after random fracture. Commercially manufactured endonucleases can be used to make fragments of full-length genes, and exonucleases can be used according to standard procedures. For example, enzymes such as Bal3I or site-directed mutagenesis can be used to systematically remove nucleotides from the end of these genes. A variety of restriction endonucleases can also be used to obtain genes that encode active fragments. Proteases can also be used to obtain active fragments of these toxins directly.

In the present invention, equivalent proteins and/or genes encoding these equivalent proteins can be derived from B.t. isolates and/or DNA libraries. There are various ways to obtain the pesticidal proteins of the present invention. For example, antibodies of pesticidal proteins disclosed and claimed by the present invention can be used to identify and isolate other proteins from a mixture of proteins. In particular, antibodies may be produced by the most constant and the most different parts from other B.t. proteins. By immunoprecipitation, enzyme-linked immunosorbent assay (ELISA) or western blot, these antibodies can be used to specifically identify equivalent proteins with characteristic activities. Standard procedures in the art can be used to prepare antibodies of the proteins or equivalents or fragments thereof disclosed in the present invention. Also, the genes encoding these proteins can be obtained from microorganisms.

Due to the redundance of genetic codes, a variety of different DNA sequences can encode the same amino acid sequence. The skilled person in the art would be able to generate alternative DNA sequences to encode the same or substantially the same protein. These different DNA sequences are included within the scope of the present invention. The “substantially the same” sequences including fragments with pesticidal activity, refer to sequences with amino acid substitution, deletion, addition or insertion but the pesticidal activity thereof is not essentially affected.

In the present invention, the substitutions, deletions or additions in amino acid sequences are conventional techniques in the art. Preferably, the alterations of amino acid sequences are: a slight change of characteristics, i.e., conservative amino acid substitutions that do not significantly affect folding and/or activity of proteins; a short deletion, usually of 1-30 amino acids; a small amino- or carboxyl-terminal extension, such as an amino-terminal extension of a methionine residue; a small peptide linker with a length of about 20-25 residues for example.

Examples of conservative substitutions are selected from the following groups of amino acids: basic amino acids, such as arginine, lysine and histidine; acidic amino acids, such as glutamic acid and aspartic acid; polar amino acids, such as glutamine, asparagine; hydrophobic amino acids, such as leucine, isoleucine and valine; aromatic amino acids, such as phenylalanine, tryptophan and tyrosinel; and small-molecule amino acids, such as glycine, alanine, serine, threonine and methionine. Typically, the amino acid substitutions without changing specific activities are well known in the art, and they have been, for example, described in “Protein” by N. Neurath and R. L. Hill in 1979, published by Academic Press, New York. The most common substitutions are Ala/Ser, Val/Ile, Asp/Glu, Thu/Ser, Ala/Thr, Ser/Asn, Ala/Val, Ser/Gly, Tyr/Phe, Ala/Pro, Lys/Arg, Asp/Asn, Leu/Ile, Leu/Val, Ala/Glu and Asp/Gly, and opposite substitutions thereof.

It would be obvious for the skilled person in the art that, these substitutions may occur outside the regions that play important roles on the molecular functions and still produce an active polypeptide. For the polypeptides according to the present invention, amino acid residues that are necessary for their activity are not selected for substitution, and that can be identified through the methods known in the art, such as site-directed mutagenesis or alanine scanning mutagenesis (referring to Cunningham and Wells, 1989, Science 244: 1081-1085). The latter technique is to introduce mutation(s) to each positive charged residue in a molecule and detect pest-resistant activity of the resulting mutants, and then to determine which amino acid residues are important for the activity of the molecule. Substrate-enzyme interaction sites can be identified by the analysis of their three-dimensional structures which can be determined by nuclear magnetic resonance analysis, crystallography or photoaffinity labeling, etc (referring to de Vos et al., 1992, Science 255: 306-312; Smith et al., 1992, J. Mol. Biol. 224: 899-904; Wlodaver et al., 1992, FEBS Letters 309: 59-64).

In the present invention, the Cry1A proteins include, but not limited to, Cry1Ab, Cry1Ah, Cry1A. 105 and Cry1Ac proteins, pesticidal fragments or functional regions that are at least 70% homologous to the amino acid sequences of the above-mentioned proteins and have the pesticidal activity to Conogethes punctiferalis.

Therefore, amino acid sequences with certain homology to SEQ ID NO: 1, 2 and/or 3 are also included in the present invention. Typically, the homology/similarity/identity of these sequences to the sequences of the present invention is greater than 60%, preferably greater than 75%, more preferably greater than 80%, even more preferably greater than 90% and can be greater than 95%. Also, preferred polynucleotides and proteins of the present invention can be defined by a more particular range of identity and/or similarity and/or homology, for example, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% identity and/or similarity and/or homology to the exemplified sequences of the present invention.

The regulatory sequences described in the present invention include, but not limited to, promoters, transit peptides, terminators, enhancers, leader sequences, introns and other regulatory sequences that can be operatively linked to Cry1A protein.

The promoters are those expressible in plants; the “promoters expressible in plants” refer to the promoters that ensure expression of the coding sequences connected thereto in plant cells. The promoters expressible in plants can be constitutive promoters. Examples of the promoters directing constitutive expression in the plants include, but not limited to, 35S promoter from the cauliflower mosaic virus, Ubi promoter, promoter of rice GOS2 gene, etc. Alternatively, the promoters expressible in plants can be tissue-specific, i.e., the expression of coding sequences directed by such promoters in some plant tissues, such as green tissues, is higher than that in other tissues, as determined by routine RNA tests, e.g., PEP carboxylase promoter. Alternatively, the promoters expressible in plants can be wound-inducible promoters. Wound-inducible promoters or promoters directing wound-induced expression patterns refer to that the expression of coding sequences regulated by such promoters is significantly higher in the plants that suffer from mechanical wound or wound caused by pest chewing than in plants under normal growth conditions. Examples of the wound inducible promoters include, but not limited to, promoters of protease inhibitor genes of potato and tomato (pin I and pin II) and of protease inhibitor gene of maize (MPI).

The transit peptides, also known as secretory signal sequences or guide sequences, can direct transgenic products to specific organelles or cellular compartments. The transit peptides can be heterologous for the target proteins. For example, the sequences encoding the chloroplast transit peptide are used to target chloroplast, or ‘KDEL’ retaining sequences are used to target endoplasmic reticulum, or using CTPP of barley lectin gene are used to target vacuoles.

The leader sequences include, but not limited to, leader sequences of small RNA viruses, such as EMCV leader sequence (5′-terminal noncoding region of EMCV (encephalomyocarditis virus)); potyvirus leader sequences, such as MDMV (maize dwarf mosaic virus) leader sequence; human immunoglobulin heavy-chain binding protein (BiP); untranslated leader sequence of mRNA of coat protein of alfalfa mosaic virus (AMV RNA4); and tobacco mosaic virus (TMV) leader sequence.

The enhancers include, but not limited to, cauliflower mosaic virus (CaMV) enhancer, figwort mosaic virus (FMV) enhancer, carnation efflorescence ring virus (CERV) enhancer, cassava vein mosaic virus (CsVMV) enhancer, mirabilis mosaic virus (MMV) enhancer, cestrum yellow leaf curl virus (CmYLCV) enhancer, cotton leaf curl Multan virus (CLCuMV) enhancer, commelina yellow mottle virus (CoYMV) enhancer and peanut chlorotic leaf streak virus (PCLSV) enhancer.

For applications in the monocotyledon, introns include, but not limited to, maize hsp70 intron, maize ubiquitin intron, Adh intron 1, sucrose synthase intron or rice Act1 intron. For applications in the dicotyledon, introns include, but not limited to, CAT-1 intron, pKANNIBAL intron, PIV2 intron and “super ubiquitin” intron.

The terminators can be signal sequences suitable for polyadenylation and functioning in plants, include but not limited to, polyadenylation signal sequences derived from nopaline synthase (NOS) gene of Agrobacterium tumefaciens, from protease inhibitor II (pin II) gene, from pea ssRUBISCO E9 gene and from α-tubulin gene.

The “effective connections” described in the present invention means the connections of nucleic acid sequences and the connections allow sequences to provide desired functions for connected sequences. The “effective connections” described in the present invention may be the connection between promoters and sequences of interest, and whereby the transcription of the sequences of interest is controlled and regulated by the promoters. When the sequences of interest encode proteins and the expression of the proteins is desired, the “effective connections” means the promoters are connected with the sequences in such a way that makes the resulting transcripts translated with a high efficiency. If the connections of the promoters and the coding sequences result in fusion transcripts and the expression of the encoded proteins is desired, such connections allow that the start codon of the resulting transcripts is the initial codon of the coding sequences. Alternatively, if the connections of promoters and coding sequences result in fusion translations and the expression of the proteins is desired, such connections allow the first start codon contained in the 5′ untranslated sequences to be connected with the promoters, and the resulted translation products to be in frame relative to the open reading frames of the desired proteins. Nucleic acid sequences for “effective connections” include, but not limited to, sequences providing genes with expression function, i.e., gene expression elements, such as promoters, 5′ untranslated region, introns, protein-coding regions, 3′ untranslated regions, polyadenylation sites and/or transcription terminators; sequences providing DNA transfer and/or integration, i.e., T-DNA border sequences, recognition sites of site-specific recombinase, integrase recognition sites; sequences providing selection, i.e., antibiotic resistance markers, biosynthetic genes; sequences providing a scoring markers and assisting operations in vitro or in vivo, i.e., multilinker sequences, site-specific recombination sequences; and sequences providing replication, i.e., bacterial origins of replication, autonomously replicating sequences and centromere sequences.

The “pesticide” described in the present invention means that it is toxic to crop pests, and more specifically to Conogethes punctiferalis.

In the present invention, Cry1A protein exhibits cytotoxicity to Conogethes punctiferalis. The transgenic plants, especially the maize and sorghum, in which their genomes contain exogenous DNA comprising nucleotide sequences encoding Cry1A protein, can lead to growth suppression and eventual death of Conogethes punctiferalis by their contact with the protein after ingestion of plant tissues. Suppression is lethal or sub-lethal. Meanwhile, the plants should be morphologically normal and can be cultured by conventional methods for the consumption and/or generation of the product. In addition, the transgenic plants can basically terminate the usage of chemical or biological pesticides that are Cry1A-targeted for Conogethes punctiferalis.

The expression level of pesticidal crystal proteins (ICP) in plant tissues can be determined by a variety of methods in the art, e.g., quantification of mRNA encoding the pesticidal proteins by specific primers, or direct quantification of pesticidal proteins.

Various tests can be applied for determining the pesticidal effects of ICP in plants. The main target of this present invention is Conogethes punctiferalis.

In the present invention, the Cry1A protein may have the amino acid sequences shown as SEQ ID NO: 1, SEQ ID NO: 2 and/or SEQ ID NO: 3 in the sequence list. In addition to Cry1A protein coding region, other components, such as regions encoding a selection marker protein, can be contained.

Moreover, the expression cassette comprising the nucleotide sequence encoding Cry1A protein of the present invention can simultaneously express at least one more genes encoding herbicide resistance proteins. The herbicide resistance genes include, but not limited to, glufosinate resistance genes, such as bar gene and pal gene; phenmedipham resistance genes, such as pnph gene; glyphosate resistance genes, such as EPSPS gene; bromoxynil resistance genes; sulfonylurea resistance genes; herbicide dalapon resistance genes; cyanamide resistance genes; or glutamine synthetase inhibitor resistance genes such as PPT, thereby obtaining transgenic plants having both high pesticidal activity and herbicide resistance.

In the present invention, foreign DNA is introduced into plants; for example, the genes or expression cassettes or recombinant vectors encoding Cry1A protein are introduced into plant cells. Conventional transformation methods include, but not limited to, Agrobacterium-mediated transformation, micro-emitting bombardment, direct DNA uptake of protoplasts, electroporation, or silicon whisker mediated DNA introduction.

The present invention provides a method for controlling pests, with the following advantages:

I. Internal control. Existing technologies are mainly through external actions, i.e. external factors to control the infestation of Conogethes punctiferalis, such as agricultural control and chemical control. While the present invention is through Cry1A produced in plants to kill Conogethes punctiferalis and subsequently control Conogethes punctiferalis, i.e., through internal factors to control.
II. No pollution and no residue. The chemical control in the art plays a certain role in the control of Conogethes punctiferalis, but it brings pollution, destruction and residues to people, livestock and farmland ecosystem. The method for controlling Conogethes punctiferalis of the present invention can eliminate the above adverse consequences.
III. Control throughout the growth period. The methods for controlling Conogethes punctiferalis in the art are staged, while the present invention provides plants with the protection throughout their growth period. That is, the transgenic plants (with Cry1A) from germination, growth, until flowering, fruiting can avoid the damage from Conogethes punctiferalis.
IV. Control of whole individual plants. The methods for controlling Conogethes punctiferalis in the art, for example foliar spray, are mostly localized. While the present invention provides a protection for the whole individual plants, for example, the roots, leaves, stems, tassels, ears, anthers, filaments, etc. of the individual transgenic plants (with Cry1A) are resistant to Conogethes punctiferalis.
V. Stable effects. The current methods of pesticide spray require direct spraying to the surface of the crops, causing the degradation of active crystal protein (including Cry1A protein) in the environment. The present invention generates plants expressing Cry1A protein with stable level in vivo, which avoids the deficiency of instability of pesticides in the environment. Also, the transgenic plants (Cry1A protein) have a consistently stable effect of controlling in different locations, different time and different genetic backgrounds.
VI. Simple, convenient, economical. Biological pesticides used in the art are easily degraded in the environment, therefore need repeated manufacturing and repeated application, and bring difficulties to agricultural production in the practical application, thus greatly increase the cost. In contrast, the present invention only need to plant transgenic plants that express Cry1A protein, thus it saves a lot of manpower, materials and financial resources.
VII. Complete effect. Methods for controlling Conogethes punctiferalis in the art are not completely efficient, and only slightly reduce the damage. In contrast, the transgenic plants (with Cry1A) in the present invention can lead to massive death of the newly hatched larvae of Conogethes punctiferalis, and can cause great progress suppression of small part of survived larvae. After 3 days, the larvae are still in the newly hatched state, and they are evidently underdeveloped and have stopped development, the transgenic plants are generally subject to minor damage.

The present invention will be described in details through the following drawings and examples.

### BRIEF DESCRIPTION OF THE DRAWINGS

FIG. 1 is a flow diagram for constructing recombinant cloning vector DBN01-T comprising the nucleotide sequence of Cry1Ab-01 in the pest control method of the present invention;

FIG. 2 is a flow diagram for constructing recombinant expression vector DBN1000124 comprising the nucleotide sequence of Cry1Ab-01 in the pest control method of the present invention;

FIG. 3 shows damages to leaves of the transgenic maize plants with inoculation of Conogethes punctiferalis in the pest control method of the present invention;

FIG. 4 shows the development of Conogethes punctiferalis larvae that are inoculated to the transgenic maize plants in the pest control method of the present invention.

### DETAILED DESCRIPTION

### Examples

Examples illustrating the method for pest control of the present invention are described as follows.

### Example 1

### Acquisition and Synthesis of Cry1A Gene

### I. Acquiring the Nucleotide Sequences of Cry1A

The amino acid sequence (818 amino acids) of pesticidal protein Cry1Ab-01 is shown as SEQ ID NO: 1 in the sequence list; the nucleotide sequence (2457 nucleotides) of Cry1Ab-01 encoding said amino acid sequence (818 amino acids) of pesticidal protein Cry1Ab-01 is shown as SEQ ID NO: 4 in the sequence list. The amino acid sequence (615 amino acids) of pesticidal protein Cry1Ab-02 is shown as SEQ ID NO: 2 in the sequence list; the nucleotide sequence (1848 nucleotides) of Cry1Ab-02 encoding the amino acid sequence (615 amino acids) of pesticidal protein Cry1Ab-02 is shown as SEQ ID NO: 5 in the sequence list.

The amino acid sequence (699 amino acids) of pesticidal protein Cry1Ah is shown as SEQ ID NO: 3 in the sequence list; the nucleotide sequence (2100 nucleotides) of Cry1Ah-01 encoding said amino acid sequence (699 amino acids) of pesticidal protein Cry1Ah is shown as SEQ ID NO: 6 in the sequence list; the nucleotide sequence (2100 nucleotides) of Cry1Ah-02 encoding the amino acid sequence (699 amino acids) of pesticidal protein Cry1Ah is shown as SEQ ID NO: 7 in the sequence list.

### II. Acquiring Nucleotide Sequences of Cry-Like Genes

The nucleotide sequence (1818 nucleotides) of Cry1Fa encoding the amino acid sequence (605 amino acids) of pesticidal protein Cry1Fa, is shown as SEQ ID NO: 8 in the sequence list; the nucleotide sequence (1947 nucleotides) of Cry1Ie encoding the amino acid sequence (648 amino acids) of pesticidal protein Cry1Ie, is shown as SEQ ID NO: 9 in the sequence list.

### III. Synthesizing the Above-Mentioned Nucleotide Sequences

The nucleotide sequences of Cry1Ab-01 (shown as SEQ ID NO: 4 in the sequence list), Cry1Ab-02 (shown as SEQ ID NO: 5 in the sequence list), Cry1Ah-01 (shown as SEQ ID NO: 6 in the sequence list), CryAh-02 (shown as SEQ ID NO: 7 in the sequence list), Cry1Fa (shown as SEQ ID NO: 8 in the sequence list) and Cry1Ie (shown as SEQ ID NO: 9 in the sequence list) are synthesized by Nanjing GenScript Ltd. 5′ end of the synthesized nucleotide sequence of Cry1Ab-01 (SEQ ID NO: 4) is connected to restriction site of NcoI, 3′ end of the synthesized nucleotide sequence of Cry1Ab-01 (SEQ ID NO: 4) is connected to restriction site of SpeI; 5′ end of the synthesized nucleotide sequence of Cry1Ab-02 (SEQ ID NO: 5) is connected to restriction site of NcoI, 3′ end of the synthesized nucleotide sequence of Cry1Ab-02 (SEQ ID NO: 5) is connected to restriction site of SwaI; 5′ end of the synthesized nucleotide sequence of Cry1Ah-01 (SEQ ID NO: 6) is connected to restriction site of AscI, 3′ end of the synthesized nucleotide sequence of Cry1Ah-01 (SEQ ID NO: 6) is connected to restriction site of SpeI; 5′ end of the synthesized nucleotide sequence of Cry1Ah-02 (SEQ ID NO: 7) is connected to restriction site of AscI, 3′ end of the synthesized nucleotide sequence of Cry1Ah-02 (SEQ ID NO: 7) is connected to restriction site of SpeI; 5′ end of the synthesized nucleotide sequence of Cry1Fa (SEQ ID NO: 8) is connected to restriction site of AscI, 3′ end of the synthesized nucleotide sequence of Cry1Fa (SEQ ID NO: 8) is connected to restriction site of BamHI; 5′ end of the synthesized nucleotide sequence of Cry1Ie (SEQ ID NO: 9) is connected to restriction site of NcoI, 3′ end of the synthesized nucleotide sequence of Cry1Ie (SEQ ID NO: 9) is connected to restriction site of SwaI.

### Example 2

### Construction of Recombinant Expression Vectors and Transformation the Same into Agrobacterium

### I. Constructing Recombinant Cloning Vectors Comprising Cry1A Gene

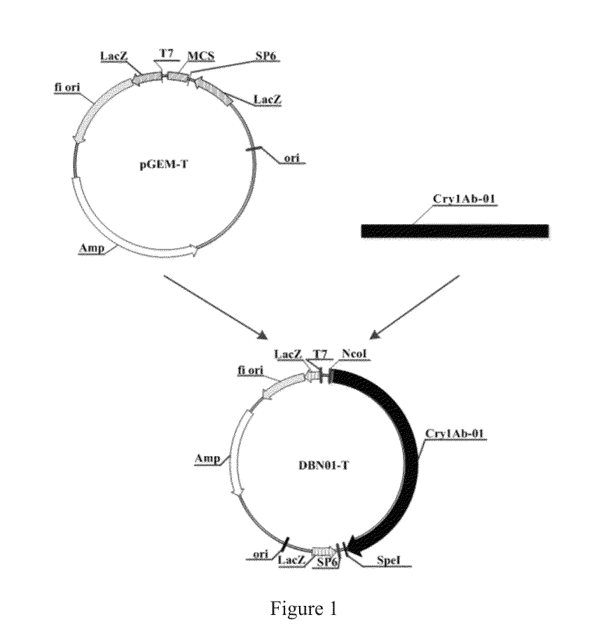

As shown in FIG. 1, the synthesized nucleotide sequence of Cry1Ab-01 was ligated with cloning vector pGEM-T (Promega, Madison, USA, CAT: A3600) according to manufacturer's protocol to generate the recombinant cloning vector DBN01-T. (Note: Amp represents Ampicillin resistance gene; f1 represents the replication origin of phage f1; LacZ is the start codon of LacZ; SP6 is the promoter of SP6 RNA polymerase; T7 is the promoter of T7 RNA polymerase; Cry1Ab-01 is the nucleotide sequence of Cry1Ab-01 (SEQ ID NO: 4); and MCS is a multi-cloning site).

The next step was to transform the recombinant cloning vector DBN01-T into competent cells T1 of E. coli (Transgen, Beijing, China, CAT: CD501) through a heat-shock method. Specifically, 50 μl competent cells T1 of E. coli were mixed with 10 μl plasmid DNA (the recombinant cloning vector DBN01-T), incubated in a water bath at 42° C. for 30 seconds and then in a water bath at 37° C. for 1 hour (in a shaker at 100 rpm). The mixture was then grew overnight on a LB plate (tryptone 10 g/L, yeast extract 5 g/L, NaCl 10 g/L, agar 15 g/L, the pH value was adjusted to 7.5 with NaOH) with Ampicillin (100 mg/l), of which the surface was coated with IPTG (isopropyl-thio-β-D-galactoside) and X-gal (5-bromo-4-chloro-3-indolyl-β-D-galactoside). White colonies were picked up and cultured further at 37° C. overnight in LB medium (tryptone 10 g/L, yeast extract 5 g/L, NaCl 10 g/L, Ampicillin 100 mg/L, pH value was adjusted to 7.5 with NaOH). The plasmids were extracted by an alkaline method. Specifically, the cultured bacteria in the medium were centrifuged at 12000 rpm for 1 min. The supernatant was discarded and the precipitated cells were resuspended in 100 μl ice-cold solution 1 (25 mM Tris-HCl, 10 mM EDTA (ethylenediamine tetraacetic acid), 50 mM glucose, pH8.0). Following the addition of 150 μl of freshly prepared solution II (0.2 M NaOH, 1% SDS (sodium dodecyl sulfate)), the tube was inverted for four times and placed on ice for 3-5 min. 150 μl ice-cold solution III (4 M potassium acetate, 2 M acetic acid) was added to the mixture, mixed immediately and thoroughly and then placed on ice for 5-10 min, followed by a centrifuge at 12000 rpm for 5 min at 4° C. The supernatant was added into 2 volumes of anhydrous ethanol, mixed thoroughly and then incubated for 5 min at room temperature. The mixture was centrifuged at 12000 rpm for 5 min at 4° C. and the supernatant was discarded. The pellet was washed with 70% (V/V) ethanol and then air dried, followed by adding 30 μl of TE (10 mM Tris-HCl, 1 mM EDTA, PH 8.0) containing RNase (20 μg/ml) to dissolve the pellet and digesting RNA in a water bath at 37° C. for 30 min. The plasmids obtained were stored at −20° C. before use.

The extracted plasmids were identified by KpnI and BglI digestion, and positive clones were further verified by sequencing. The results showed that, the nucleotide sequence inserted into the recombinant cloning vector DBN01-T was the nucleotide sequence of Cry1Ab-01 shown as SEQ ID NO: 4 in the sequence list, indicating the proper insertion of the nucleotide sequence of Cry1Ab-01.

As the above-mentioned method for the construction of the recombinant cloning vector DBN01-T, the synthesized nucleotide sequence of Cry1Ab-02 (shown as SEQ ID NO: 5) was ligated with cloning vector pGEM-T to generate the recombinant cloning vector DBN02-T. The proper insertion of the nucleotide sequence Cry1Ab-02 in the recombinant vector DBN02-T was verified by enzymatic digestion and sequencing.

As the above method for the construction of the recombinant cloning vector DBN01-T, the synthesized nucleotide sequence of Cry1Ah-01 (shown as SEQ ID NO: 6) was ligated with cloning vector pGEM-T to generate the recombinant cloning vector DBN03-T. The proper insertion of the nucleotide sequence Cry1Ah-01 in the recombinant vector DBN03-T was verified by enzymatic digestion and sequencing.

As the above method for the construction of the recombinant cloning vector DBN01-T, the synthesized nucleotide sequence of Cry1Ah-02 (shown as SEQ ID NO: 7) was ligated with cloning vector pGEM-T to generate the recombinant vector DBN04-T. The proper insertion of the nucleotide sequence Cry1Ah-02 in the recombinant vector DBN04-T was verified by enzymatic digestion and sequencing.

As the above method for the construction of the recombinant cloning vector DBN01-T, the synthesized nucleotide sequence of Cry1Fa (shown as SEQ ID NO: 8) was ligated with cloning vector pGEM-T to generate the recombinant vector DBN05-T. The proper insertion of the nucleotide sequence Cry1Fa in the recombinant vector DBN05-T was verified by enzymatic digestion and sequencing.

As the above method for the construction of the recombinant cloning vector DBN01-T, the synthesized nucleotide sequence of Cry1Ie (shown as SEQ ID NO: 9) was ligated with cloning vector pGEM-T to generate the recombinant vector DBN06-T. The proper insertion of the nucleotide sequence Cry1Ie in the recombinant vector DBN06-T was verified by enzymatic digestion and sequencing.

### II. Constructing Recombinant Expression Vectors Comprising Cry1A Gene

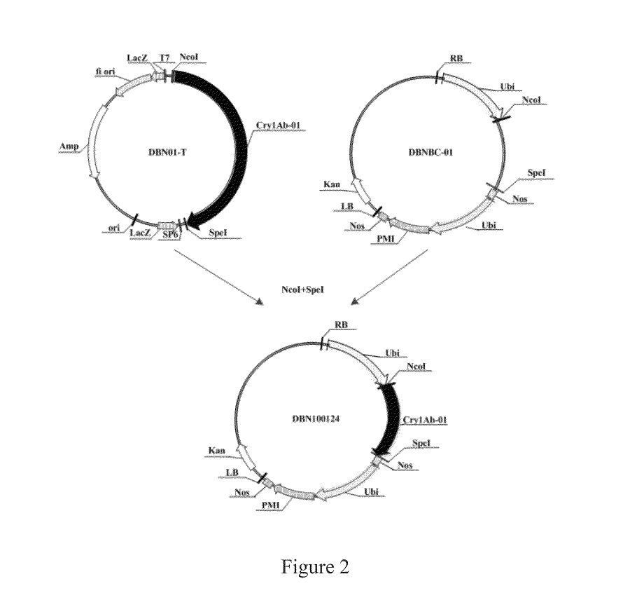

Methods for constructing vectors by conventional enzymatic digestion are well known in the art. As shown in FIG. 2, the recombinant cloning vector DBN01-T and expression vector DBNBC-01 (Vector backbone: pCAMBIA2301 (available from CAMBIA institution)) were digested respectively by the restriction enzymes NcoI and SpeI; and the resulting fragment of the nucleotide sequence of Cry1Ab-01 was then inserted into the digested expression vector DBNBC-01 between NcoI and SpeI sites to generate the recombinant expression vector DBN100124. (Note: Kan represents kanamycin gene; RB represents right border; Ubi represents the promoter of maize ubiquitin gene (SEQ ID NO: 10); Cry1Ab-01 represents the nucleotide sequence of Cry1Ab-01 (SEQ ID NO: 4); Nos represents the terminator of nopaline synthase gene (SEQ ID NO: 11); PMI represents phosphomannose isomerase gene (SEQ ID NO: 12); and LB represents left border).

The recombinant expression vector DBN100124 was transformed into competent cells T1 of E. coli through a heat-shock method. Specifically, 50 μl competent cells T1 of E. coli were mixed with 10 μl plasmid DNA (the recombinant expression vector DBN100124), incubated in a water bath at 42° C. for 30 seconds and then in a water bath at 37° C. for 1 hour (in a shaker at 100 rpm). The mixture was then grew at 37° C. for 12 hours on a LB plate (tryptone 10 g/L, yeast extract 5 g/L. NaCl 10 g/L, agar 15 g/L, the pH value was adjusted to 7.5 with NaOH) with 50 mg/L Kanamycin. White colonies were picked up and cultured further at 37° C. overnight in LB medium (tryptone 10 g/L, yeast extract 5 g/L, NaCl 10 g/L, Kanamycin 50 mg/L, the pH value was adjusted to 7.5 with NaOH). The plasmids were extracted by an alkaline method. Enzymatic digestion with NcoI and SpeI was used to identify the extracted plasmids, and positive clones were further verified by sequencing. The results showed that, the nucleotide sequence inserted into the recombinant expression vector DBN100124 between NcoI and SpeI sites was Cry1Ab-01 shown as SEQ ID NO: 4 in the sequence list.

As the above method for the construction of the recombinant expression vector DBN100124, the recombinant cloning vector DBN02-T and DBN05-T were enzymatically digested by NcoI and SwaI, AscI and BamHI respectively to generate the nucleotide sequences of Cry1Ab-02 and Cry1Fa, which were inserted into the expression vector DBNBC-01 to obtain the recombinant expression vector DBN100075. As verified by enzymatic digestion and sequencing, the recombinant expression vector DBN100075 included the nucleotide sequences of Cry1Ab-02 and Cry1Fa shown in SEQ ID NO: 5 and SEQ ID NO: 8 in the sequence list.

As the above method for the construction of the recombinant expression vector DBN100124, the recombinant cloning vector DBN03-T was enzymatically digested by AscI and SpeI to generate the nucleotide sequence of Cry1Ah-01, which was inserted into the expression vector DBNBC-01 to obtain the recombinant expression vector DBN100071. As verified by enzymatic digestion and sequencing, the nucleotide sequence inserted into the recombinant expression vector DBN100071 between AscI and SpeI sites was Cry1Ah-01.

As the above method for the construction of the recombinant expression vector DBN100124, the recombinant cloning vector DBN04-T and DBN06-T were enzymatically digested by AscI and SpeI, NcoI and SwaI respectively to generate the nucleotide sequences of Cry1Ah-02 and Cry1Ie, which were inserted into the expression vector DBNBC-01 to obtain the recombinant expression vector DBN100147. As verified by enzymatic digestion and sequencing, the recombinant expression vector DBN100147 included the nucleotide sequences of Cry1Ah-02 and Cry1Ie shown in SEQ ID NO: 7 and SEQ ID NO: 9 in the sequence list.

III. Recombinant Expression Vectors were Transformed into Agrobacterium

The correctly constructed recombinant expression vectors of DBN100124, DBN100075, DBN100071 and DBN100147 were transformed into Agrobacterium LBA4404 (Invitrogen, Chicago, USA; Cat No: 18313-015) through a liquid nitrogen method, respectively. Specifically, 100 μL Agrobacterium LBA4404 and 3 μL plasmid DNA (the recombinant expression vector from DBN100124, DBN100075, DBN100071 and DBN100147) were placed in liquid nitrogen for 10 minutes, followed by incubation in a water bath at 37° C. for 10 minutes. The transformed Agrobacterium LBA4404 were inoculated in a LB tube and then cultured at 28° C., 200 rpm for 2 hours. Subsequently, the culture was applied to a LB plate containing 50 mg/L Rifampicin and 100 mg/L Kanamycin until positive individual clonies grew. The individual clonies were picked for further culture to extract plasmids. The recombinant expression vectors were identified by enzymatic digestion, that is, the recombinant expression vector DBN100124 and DBN100075 were digested with restriction enzymes AhdI and XbaI, and the recombinant expression vector DBN100071 and DBN100147 were digested with restriction enzymes StyI and BglII, indicating the correct construction of the recombinant expression vectors, DBN100124, DBN100075, DBN100071 and DBN100147.

### Example 3

### Acquisition and Verification of Maize Plants Transformed with Cry1A Gene

I. Generation and Identification of Maize Plants Transformed with Cry1A Gene

According to the conventional Agrobacterium infection method, the sterile cultured immature embryos of Maize Z31 were cultured with Agrobacterium strains obtained in III, Example 2, so as to transform T-DNAs in the recombinant expression vectors DBN100124, DBN100075, DBN100071 and DBN100147 (comprising the promoter sequence of maize Ubiquitin gene, the nucleotide sequences of Cry1Ab-01, Cry1Ab-02, Cry1Ah-01, Cry1Ah-02, Cry1Fa and Cry1Ie, PMI gene and the terminator sequence of Nos) into the maize genome, generating the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence of Cry1Ah-02-Cry1Ie. The wild-type maize plants were used as control.

The process of Agrobacterium-mediated transformation of maize was briefly described as follows. The immature embryos isolated from the maize were contacted with the Agrobacterium suspension, whereby the nucleotide sequences of Cry1Ab-01, Cry1Ah-02-Cry1Fa, Cry1Ah-01 and/or Cry1Ah-02-Cry1Ie were delivered into at least one cell of either immature embryo by Agrobacterium (step 1: Infection). In this step, the immature embryos were preferably immersed in Agrobacterium suspension (OD₆₆₀=0.4-0.6, infection medium (MS salt 4.3 g/L, MS vitamins, casein 300 mg/L, sucrose 68.5 g/L, glucose 36 g/L, Acetosyringone (AS) 40 mg/L, 2,4-dichlorophenoxyacetic acid (2,4-D) 1 mg/L, pH 5.3)) to initiate inoculation. The immature embryos were cultured with Agrobacterium for a period of time (3 days) (step 2: Co-culture). Preferably, after the step of infection, the immature embryos were cultured on a solid medium (MS salt 4.3 g/L, MS vitamins, casein 300 mg/L, sucrose 20 g/L, glucose 10 g/L, Acetosyringone (AS) 100 mg/L, 2,4-dichlorophenoxyacetic acid (2,4-D) 1 mg/L, agar 8 g/L, pH 5.8). After the co-culture step, a “recovery” step is optional, wherein there is at least an antibiotic known as inhibiting the growth of Agrobacterium (Cephalosporins) and no selection agents for plant transformants in the recovery medium (MS salt 4.3 g/L, MS vitamins, casein 300 mg/L, sucrose 30 g/L, 2,4-dichlorophenoxyacetic acid (2,4-D) 1 mg/L, agar 8 g/L, pH 5.8) (step 3: Recovery). Preferably, the immature embryos were cultured on the solid medium with an antibiotic but without selection agents to eliminate Agrobacterium and provide a recovery period for transformed cells. Next, the inoculated immature embryos were cultured on the medium with a selection agent (mannose) and the growing transformed calluses were selected (step 4: Selection). Preferably, the immature embryos were cultured on a solid selection medium with a selection agent (MS salt 4.3 g/L, MS vitamins, casein 300 mg/L, sucrose 5 g/L, mannose 12. 5 g/L, 2,4-dichlorophenoxyacetic acid (2,4-D) 1 mg/L, agar 8 g/L, pH 5.8), which resulted in a selective growth of transformed cells. Further, the calluses regenerated into plants (step 5: Regeneration). Preferably, the calluses grown on the medium with the selection agent were cultured on a solid medium (MS differentiation medium and MS rooting medium) to regenerate plants.

The selected resistant calluses were transferred onto the MS differentiation medium (MS salt 4.3 g/L, MS vitamins, casein 300 mg/L, sucrose 30 g/L, 6-benzyladenine 2 mg/L, mannose 5 g/L, agar 8 g/L, pH 5.8), and cultured under 25° C. for differentiation. The differentiated seedlings were transferred onto the MS rooting medium (MS salt 2.15 g/L, MS vitamins, casein 300 mg/L, sucrose 30 g/L, indole-3-acetic acid 1 mg/L, agar 8 g/L, pH 5.8), and cultured under 25° C. till the height of about 10 cm. The seedlings were then transferred into a greenhouse and grew to fructify. During the culture in the greenhouse, the seedlings were incubated at 28° C. for 16 hours and then incubated at 20° C. for 8 hours each day.

II. Verification of Maize Plants Transformed with Cry1A Gene by TaqMan Method

Using about 100 mg of the leaves from each of the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence of Cry1Ah-02-Cry1Ie as samples, the genomic DNA was extracted with DNeasy Plant Maxi Kit of Qiagen, and the copy numbers of Cry1A, Cry1F and Cry1Ie genes were determined by a fluorescence quantitative PCR assay with Taqman probe. The wild-type maize plants were analyzed as control according to the above-mentioned method. The experiments were repeated for 3 times and the results were averaged.

The detailed protocol for determining the copy number of Cry1A, Cry1F and Cry1Ie genes was as follows:

Step 11: 100 mg of the leaves from each of the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence of Cry1Ah-02-Cry1Ie, and that of the wild-type maize plants were sampled and homogenized in a mortar with liquid nitrogen. Each sample was in triplicate.

Step 12: The genomic DNA of the above-mentioned samples was extracted with DNeasy Plant Maxi Kit of Qiagen, and the detailed method refers to the manufacturer's protocol.

Step 13: NanoDrop 2000 (Thermo Scientific) was employed to measure genomic DNA concentrations of the above-mentioned samples.

Step 14: The concentrations of genomic DNA of the above-mentioned samples were adjusted to the same in a range of 80-100 ng/μl.

Step 15: The copy numbers of the samples were determined by a fluorescence quantitative PCR method with Taqman probe. A sample that had a known copy number was used as standard, and a sample from the wild-type maize plants was used as control. Each sample was triplicated and the results were averaged. The primers and probes used in the fluorescence quantitative PCR method are as follows.

The following primers and probes were used for detecting the nucleotide sequence of Cry1Ab-01:

Primer 1 (CF1): CGAACTACGACTCCCGCAC, shown as SEQ ID NO: 13 in the sequence list;
Primer 2 (CR1): GTAGATTTCGCGGGTCAGTTG, shown as SEQ ID NO: 14 in the sequence list;
Probe 1 (CP1): CTACCCGATCCGCACCGTGTCC, shown as SEQ ID NO: 15 in the sequence list.

The following primers and probes are used for detecting the nucleotide sequence of Cry1Ab-02:

Primer 3 (CF2): TGCGTATTCAATTCAACGACATG, shown as SEQ ID NO: 16 in the sequence list;
Primer 4 (CR2): CTTGGTAGTTCTGGACTGCGAAC, shown as SEQ ID NO: 17 in the sequence list;
Probe 2 (CP2): CAGCGCCTTGACCACAGCTATCCC, shown as SEQ ID NO: 18 in the sequence list;

The following primers and probes are used for detecting the nucleotide sequence of Cry1Fa:

Primer 5 (CF3): CAGTCAGGAACTACAGTTGTAAGAGGG, shown as SEQ ID NO: 19 in the sequence list;
Primer 6 (CR3): ACGCGAATGGTCCTCCACTAG, shown as SEQ ID NO: 20 in the sequence list;
Probe 3 (CP3): CGTCGAAGAATGTCTCCTCCCGTGAAC, shown as SEQ ID NO: 21 in the sequence list;

The following primers and probes are used for detecting the nucleotide sequence of Cry1Ah-01:

Primer 7 (CF4): ATCGTGAACAACCAGAACCAGTG, shown as SEQ ID NO: 22 in the sequence list;
Primer 8 (CR4): CTCCAGGATCTCGATCTCCG, shown as SEQ ID NO: 23 in the sequence list;
Probe 4 (CP4): CGTGCCGTACAACTGCCTGAACAACC, shown as SEQ ID NO: 24 in the sequence list;

The following primers and probes are used for detecting the nucleotide sequence of Cry1 Ah-02:

Primer 9 (CF5): TCATTTGGGGCTTCGTCG, shown as SEQ ID NO: 25 in the sequence list;
Primer 10 (CR5): TGATTGATCAGCTGCTCAACCT, shown as SEQ ID NO: 26 in the sequence list;
Probe 5 (CP5): CCAGTGGGATGCGTTCCTCGCTC, shown as SEQ ID NO: 27 in the sequence list;

The following primers and probes are used for detecting the nucleotide sequence of Cry1Ie:

Primer 11 (CF6): GAGCATTGATCCTTTCGTCAGTG, shown as SEQ ID NO: 28 in the sequence list;
Primer 12 (CR6): CAAAGTACCGAGGATCTTACCAGC, shown as SEQ ID NO: 29 in the sequence list;
Probe 6 (CP6): CCTCCACAATCCAAACGGGCATCG, shown as SEQ ID NO: 30 in the sequence list.

PCR Reaction System:

| | |
|---|---|
| JumpStart ™ Taq ReadyMix ™ (Sigma) | 10 μl |
| 50× mixture of primers/probes | 1 μl |
| Genomic DNA | 3 μl |
| Water (ddH₂O) | 6 μl |

The 50× mixture of primers/probes, containing 45 μl of 1 mM each primer, 50 μl of 100 μM probe and 860 μl of 1×TE buffer, was stored in an amber tube at 4° C.

PCR Conditions were as Follows:

| | | |
|---|---|---|
| Step | Temperature | Time |

| | | | |
|---|---|---|---|
| 21 | 95° C. | 5 | min |
| 22 | 95° C. | 30 | sec |
| 23 | 60° C. | 1 | min |

| | |
|---|---|
| 24 | returning to step 22, repeating 40 times |

The data were analyzed by SDS2.3 software (Applied Biosystems).

As shown by the results, the nucleotide sequences of Cry1Ab-01, Cry1Ab-02-Cry1Fa, Cry1Ah-01 and Cry1Ah-02-Cry1Ie were all integrated into the genome of the detected maize plants. The maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 as well as the maize plants transformed with the nucleotide sequence Cry1Ah-02-Cry1Ie had obtained a single copy of Cry1A, Cry1F and/or Cry1Ie gene in the respective transgenic maize plants.

### Example 4

### Detection of Pesticidal Proteins in the Transgenic Maize Plants

### I. Detection of the Pesticidal Protein Contents in the Transgenic Maize Plants

Solutions involved in this experiment are as follows:

Extraction buffer: 8 g/L NaCl, 0.2 g/L KH₂PO₄, 2.9 g/L Na₂HPO₄.12H₂O, 0.2 g/L KCl, 5.5 ml/L Tween-20, pH 7.4;
Washing buffer PBST: 8 g/L NaCl, 0.2 g/L KH₂PO₄, 2.9 g/L Na₂HPO₄.12H₂O, 0.2 g/L KCl, 0.5 ml/L Tween-20, pH 7.4;
Termination solution: 1M HCl.

3 mg of fresh leaves from each of the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 as well as the maize plants transformed with the nucleotide sequence Cry1Ah-02-Cry1Ie were sampled and homogenized with liquid nitrogen, followed by the addition of 800 μl extraction buffer. The mixture was centrifuged at 4000 rpm for 10 min, then the supernatant was diluted 40-fold with the extraction buffer and 80 μl of diluted supernatant was used for ELISA test. Due to the high identity between Cry1Ah amino acid sequence and Cry1Ab amino acid sequence, the antibody against Cry1Ab can be applied for pesticidal protein Cry1Ah. ELISA (enzyme-linked immunosorbent assay) kit (ENVIRLOGIX Company, Cry1Ab/Cry1Ac kit) was employed to determine the ratio of the pesticidal protein (Cry1Ab and Cry1Ah) content divided by the weight of the fresh leaves. The detailed method refers to the manufacturer's protocol.

Meanwhile, the wild-type maize plants and the non-transgenic maize plants identified by Taqman were used as controls, and the determination followed the above-described methods. For three lines transformed with Cry1Ab-01 (S1, S2 and S3), three lines transformed with Cry1Ab-01-Cry1Fa (S4, S5 and S6), three lines transformed with Cry1Ah-01 (S7, S8 and S9), three lines transformed with Cry1Ah-02-Cry1Ie (S10, S11 and S12), one line identified as non-transgenic plant (NGM) by Taqman and one line as wild type (CK), three plants for each line were used and each plant was repeated six times.

Experimental results of the pesticidal protein (Cry1Ab protein) contents in the transgenic plants were shown in Table 1. Experimental results of the pesticidal protein (Cry1Ah protein) contents in the transgenic plants were shown in Table 2. The ratios of the averaged expressions of the pesticidal protein (Cry1Ab) divided by the weight of the fresh leaves in the maize plants transformed with the nucleotide sequence of Cry1Ab-01 and Cry1Ab-01-Cry1Fa were determined as 8536.2 and 8234.7, respectively; the ratios of the averaged expressions of the pesticidal protein (Cry1Ah) divided by the weight of the fresh leaves in the maize plants transformed with the nucleotide sequence of Cry1Ah-01 and Cry1Ah-02-Cry1Ie were determined as 5374.3 and 5382.2, respectively. These results indicate that Cry1Ab and Cry1Ah proteins present a high and stable expression in the transgenic maize plants.

TABLE 1
Average amount of Cry1Ab protein expressed
in the transgenic maize plants

| | Amount of Cry1Ab protein | | | Amount of Cry1Ab protein |
|---|---|---|---|---|
| | expressed in each plant (ng/g) | | | expressed in each kind |
| | (repeated six times per plant) | | | of lines (ng/g) |
| Line | 1 | 2 | 3 | Average amount (ng/g) |
| S1 | 7160.2 | 10444.4 | 9080.8 | 8536.2 |
| S2 | 8534.4 | 8581.2 | 7330.2 | |
| S3 | 8817.4 | 9185.7 | 7691.2 | |
| S4 | 7088.4 | 9837.5 | 10626.4 | 8234.7 |
| S5 | 9866.7 | 6863.3 | 4222.4 | |
| S6 | 9912.1 | 7724.1 | 7970.9 | |
| NGM | −1.7 | 0 | −1.0 | 0 |
| CK | 0 | −4.2 | 2.3 | 0 |

TABLE 2
Average amount of Cry1Ah protein expressed
in the transgenic maize plants

| | Amount of Cry 1 Ah protein | | | Amount of Cry1Ah protein |
|---|---|---|---|---|
| | expressed in single plant (ng/g) | | | expressed in each kind |
| | (repeated six times per plant) | | | of lines (ng/g) |
| line | 1 | 2 | 3 | Average amount (ng/g) |
| S7 | 5220.5 | 5520.2 | 5550.6 | 5374.3 |
| S8 | 5130.4 | 5249.3 | 5486.5 | |
| S9 | 5205.4 | 5437.5 | 5568.1 | |
| S10 | 5305.3 | 5374.9 | 5653.8 | 5382.2 |
| S11 | 5136.0 | 5229.5 | 5546.8 | |
| S12 | 5240.3 | 5502.2 | 5450.7 | |
| NGM | −12.68 | 0 | −11.26 | 0 |
| CK | 0 | −15.13 | −13.21 | 0 |

### II. Detection of Pest Resistance of the Transgenic Maize Plants

The maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01, the maize plants transformed with the nucleotide sequence Cry1Ah-02-Cry1Ie, the wild-type maize plants and the non-transgenic maize plants identified by Taqman were detected for their resistance to Conogethes punctiferalis.

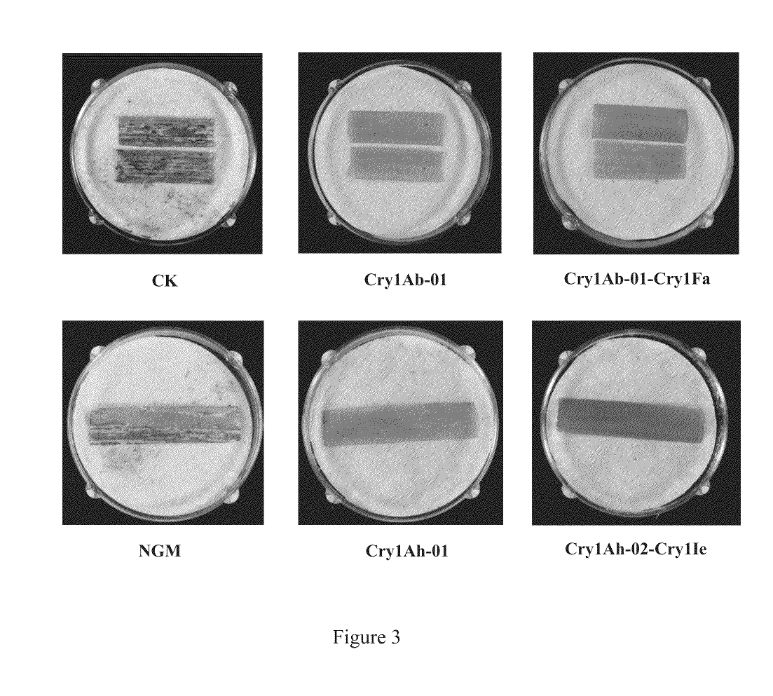

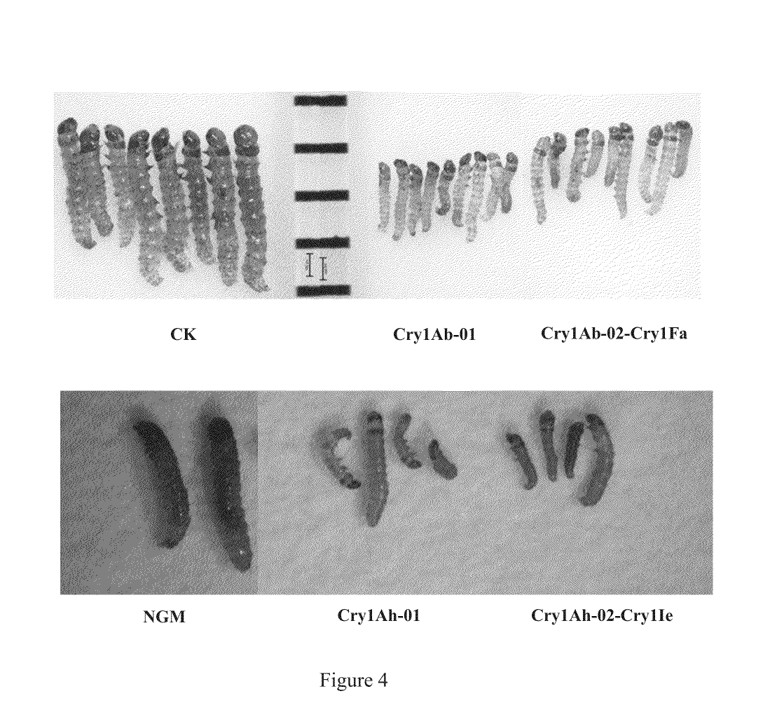

Fresh leaves of the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01, the maize plants transformed with the nucleotide sequence Cry1Ah-02-Cry1Ie, the wild-type maize plants and the maize plants identified as non-transgenic plants (V3-V4 stage) by Taqman were sampled, respectively. The leaves were rinsed with sterile water and water on the leaves was dried up by gauze. The veins of the leaves were removed, and the leaves were cut into stripes of approximately 1 cm×4 cm or 1 cm×2 cm. One or two stripes of the leaves were placed on filter paper wetted with distilled water on the bottom of round plastic Petri dishes. Ten heads of Conogethes punctiferalis (newly hatched larvae) were putted into each dish, and the dishes with pests were covered with lids and placed at 25-28° C., relative humidity of 70%-80% and photoperiod (light/dark) 16:8 for 3 days. According to three indicators, the developmental progress, mortality and leaf damage rate of the Conogethes punctiferalis's larvae, the resistance score was acquired: score=100×mortality+[100×mortality+90×(the number of newly hatched pests/the total number of inoculated pests)+60×(the number of newly hatched−the number of negative control pests/the total number of inoculated pests)+10×(the number of negative control pests/the total number of inoculated pests)]+100×(1−leaf damage rate). For three lines transformed with the nucleotide sequence Cry1Ab-01 (S1, S2 and S3), three lines transformed with the nucleotide sequence Cry1Ab-02-Cry1Fa (S4, S5 and S6), three lines transformed with the nucleotide sequence Cry1Ah-01 (S7, S8 and S9), three lines transformed with the nucleotide sequence Cry1Ah-02-Cry1Ie (S10, S11 and S12), one line identified as non-transgenic plants (NGM) by Taqman and one line as wild type (CK), three plants for each line were used and each plant was repeated six times. The results were shown in Table 3 as well as FIGS. 3 and 4.

TABLE 3
Pest resistance of the transgenic maize ptants inoculated with Conogethes punctiferalis

| | | Developmental progress of | | | Mortality of | | | |
|---|---|---|---|---|---|---|---|---|
| | | Conogethes punctiferalis | | | Conogethes | | | |
| | | (each line) | | | punctiferalis | | | |
| | | | Newly | | (each line) | | | |
| | Leaf | | hatched- | | The total | | Score | |
| | damage | Newly | negative | ≧negative | number | Mortality | (each | |
| Line | rate (%) | hatched | control | control | of pests | (%) | line) | Average |
| S1 | 1 | 1.5 | 0 | 0 | 10 | 85 | 283 | 285 |
| S2 | 1 | 1 | 0 | 0 | 10 | 90 | 288 | |
| S3 | 0.5 | 0 | 0 | 1 | 10 | 90 | 285 | |
| S4 | 1 | 1.5 | 0 | 0 | 10 | 85 | 283 | 282 |
| S5 | 3 | 1 | 0 | 0 | 10 | 85 | 279 | |
| S6 | 1 | 1.5 | 0 | 0 | 10 | 85 | 283 | |
| S7 | 2 | 0.5 | 0 | 0 | 10 | 93 | 293 | 284 |
| S8 | 2 | 1.5 | 0 | 0 | 10 | 85 | 282 | |
| S9 | 2 | 2 | 0 | 0 | 10 | 80 | 282 | |
| S10 | 1 | 1.5 | 0 | 0 | 10 | 85 | 283 | 286 |
| S11 | 3 | 0 | 0 | 0.5 | 10 | 95 | 288 | |
| S12 | 1 | 1 | 0 | 0 | 10 | 90 | 288 | |
| NGM | 40 | 0 | 0 | 7.3 | 10 | 0 | 27 | 121 |
| CK | 50 | 0 | 0 | 7.5 | 10 | 17 | 15 | 94 |

As shown in Table 3, the scores of the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01, the maize plants transformed with the nucleotide sequence Cry1Ah-02-Cry1Ie were all around 280 or above, while the score of the wild-type maize plants was generally about 120 or less.

As shown in FIGS. 3 and 4, compared with the wild-type maize plants, the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence Cry1Ah-02-Cry1Ie killed great amount of the newly hatched Conogethes punctiferalis larvae, and greatly suppressed the growth of small amount of survived larvae so that the larvae still remained in the newly hatched state after 3 days. Additionally, the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence Cry1Ah-02-Cry1Ie only had a minor damage, presenting a very small amount of pinhole-like damages; the leaf damage rates were all about 3% or less.

Thus, it is proved that the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence Cry1Ah-02-Cry1Ie were all showed high resistance to Conogethes punctiferalis, which are sufficient to cause adverse effects on the growth of Conogethes punctiferalis so that they can be controlled.

The above results also showed that, the effective control of Conogethes punctiferalis was resulted from Cry1A protein produced in the maize plants transformed with the nucleotide sequence of Cry1Ab-01, the maize plants transformed with the nucleotide sequence of Cry1Ab-02-Cry1Fa, the maize plants transformed with the nucleotide sequence of Cry1Ah-01 and the maize plants transformed with the nucleotide sequence Cry1Ah-02-Cry1Ie. Similar transgenic plants capable of expressing Cry1A could be produced to control Conogethes punctiferalis, based on the same toxic effect of Cry1A protein on Conogethes punctiferalis. Cry1Ab proteins described in the present invention include, but not limited to, the Cry1A proteins shown in the specific embodiments by the specific sequences. The transgenic plants can also generate at least one kind of a second pesticidal protein that is different from Cry1A, e.g., Cry1Ie, Cry1Fa, Vip3A and Cry1Ba proteins.

In conclusion, the present invention can control Conogethes punctiferalis by enabling the plants to produce Cry1A protein in vivo, which is toxic to Conogethes punctiferalis. In comparison with current agricultural and chemical control methods, the method described by the present invention can control Conogethes punctiferalis throughout the growth period of the plants and provide a full protection to the plants. Additionally, the method is stable, complete, simple, convenient, economical, pollution-free and residue-free.

Finally it should be noted that, the above embodiments merely illustrate the technical solutions of the present invention and it is not limited to those, although the preferred embodiments with reference to the present invention have been described in detail, people of ordinary skill in the art should appreciate that the technical solutions of the present invention can be modified or equivalently replaced without departing from the spirit and scope of the technical solutions of the invention.

1. A method for controlling Conogethes punctiferalis, wherein the method comprises contacting Conogethes punctiferalis with Cry1A protein.

2. The method of claim 1, wherein the Cry1A protein is Cry1Ab protein or Cry1Ah protein.

3. The method of claim 2, wherein the Cry1Ab protein is present in a cell that expresses the Cry1Ab protein of a plant, and Conogethes punctiferalis contacts with the Cry1Ab protein by ingestion of the cell.

4. The method of claim 3, wherein the Cry1Ab protein is present in a transgenic plant that expresses the Cry1Ab protein, and Conogethes punctiferalis contacts with the Cry1Ab protein by ingestion of a tissue of the transgenic plant; thereafter, the growth of Conogethes punctiferalis is suppressed, and that eventually leads to Conogethes punctiferalis' death and achieves controlling damage of Conogethes punctiferalis to the plant.

5. The method of claim 2, wherein the Cry1Ah protein is present in a cell that expresses the Cry1Ah protein of a plant, and Conogethes punctiferalis contacts with the Cry1Ah protein by ingestion of the cell.

6. The method of claim 5, wherein the Cry1Ah protein is present in a transgenic plant that expresses the Cry1Ah protein, and Conogethes punctiferalis contacts with the Cry1Ah protein by ingestion of a tissue of the transgenic plant; thereafter, the growth of Conogethes punctiferalis is suppressed, and that eventually leads to Conogethes punctiferalis' death and achieves controlling the damage of Conogethes punctiferalis to the plant.

7. The method of claim 4, wherein the transgenic plant is in any growth periods.

8. The method of claim 4, wherein the tissue of the transgenic plant is leaves, stems, tassels, ears, anthers or filaments.

9. The method of claim 4, wherein the control of the damage of Conogethes punctiferalis to the plant does not depend on planting location.

10. The method of claim 4, wherein the control of the damage of Conogethes punctiferalis to the plant does not depend on planting time.

11. The method of claim 4, wherein the plant is derived from maize, sorghum, millet, sunflower, castor, ginger, cotton, peach, persimmon, walnut, chestnut, fig or pine.

12. The method of claim 3, wherein prior to the step of contacting, the method comprises a step to plant a seedling that contains a polynucleotide encoding Cry1A protein.

13. The method of claim 2, wherein the amino acid sequence of Cry1A protein comprises an amino acid sequence of SEQ ID NO:1, SEQ ID NO:2 or SEQ ID NO:3.

14. The method of claim 13, the nucleotide sequence encoding Cry1A protein comprises a nucleotide sequence of SEQ ID NO:4, SEQ ID NO:5, SEQ ID NO:6 or SEQ ID NO:7.

15. The method of claim 3, wherein the plant further expresses at least one of a second nucleotide sequence, which is different from that encoding the Cry1A protein.

16. The method of claim 15, wherein the second nucleotide encodes a Cry-like pesticidal protein, a Vip-like pesticidal protein, a protease inhibitor, lectin, α-amylase or peroxidase.

17. The method of claim 16, wherein the second nucleotide encodes Cry1Ie protein, Cry1Fa protein, Vip3A protein or Cry1Ba protein.

18. The method of claim 17, wherein the second nucleotide comprises a nucleotide sequence of SEQ ID NO:8 or SEQ ID NO:9.

19. The method of claim 15, wherein the second nucleotide sequence is dsRNA, which inhibits an important gene of a target pest.

20. A transgenic plant that expresses Cry1A protein.


SEQUENCE LISTING (30 sequences)

SEQ ID NO: 1 - protein, 818 aa, Artificial
  Synthetic peptide (Cry1Ab-01 amino acid sequence)
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDFR

SEQ ID NO: 2 - protein, 615 aa, Artificial
  Synthetic peptide (Cry1Ab-02 amino acid sequence)
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEY

SEQ ID NO: 3 - protein, 699 aa, Artificial
  Synthetic peptide (Cry1Ah amino acid sequence)
MKNSIKLSELWYFNERKWRYFMEIVNNQNQCVPYNCLNNPEIEILEGGRISVGNTPIDISLSLTQFLLSEFVPGAGFVLGLIDLIWGFVGPSQWDAFLAQVEQLINQRIAEAVRNTAIQELEGMARVYRTYATAFAEWEKAPDDPELREALRTQFTATETYISGRISVLKIQTFEVQLLSVFAQAANLHLSLLRDVVFFGQRWGFSTTTVNNYYNDLTEGISTYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTFYRRPFNIGINNQQLSVLDGTEFAYGTSSNLPSAVYRKSGTVDSLDEIPPQNNNVPPRQGFSHRLSHVSMFRSGSSSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSSNQIGLKTDVTDYHIDQVSNLVECLSDEFCLDEKKELSEKVKHAKRLS

SEQ ID NO: 4 - DNA, 2457 bp, Artificial
  Synthetic oligonucleotide (Cry1Ab-01 nucleotide sequence)
atggacaacaacccaaacatcaacgagtgcatcccgtacaactgcctcagcaaccctgaggtcgaggtgctcggcggtgagcgcatcgagaccggttacacccccatcgacatctccctctccctcacgcagttcctgctcagcgagttcgtgccaggcgctggcttcgtcctgggcctcgtggacatcatctggggcatctttggcccctcccagtgggacgccttcctggtgcaaatcgagcagctcatcaaccagaggatcgaggagttcgccaggaaccaggccatcagccgcctggagggcctcagcaacctctaccaaatctacgctgagagcttccgcgagtgggaggccgaccccactaacccagctctccgcgaggagatgcgcatccagttcaacgacatgaacagcgccctgaccaccgccatcccactcttcgccgtccagaactaccaagtcccgctcctgtccgtgtacgtccaggccgccaacctgcacctcagcgtgctgagggacgtcagcgtgtttggccagaggtggggcttcgacgccgccaccatcaacagccgctacaacgacctcaccaggctgatcggcaactacaccgaccacgctgtccgctggtacaacactggcctggagcgcgtctggggccctgattctagagactggattcgctacaaccagttcaggcgcgagctgaccctcaccgtcctggacattgtgtccctcttcccgaactacgactcccgcacctacccgatccgcaccgtgtcccaactgacccgcgaaatctacaccaaccccgtcctggagaacttcgacggtagcttcaggggcagcgcccagggcatcgagggctccatcaggagcccacacctgatggacatcctcaacagcatcactatctacaccgatgcccaccgcggcgagtactactggtccggccaccagatcatggcctccccggtcggcttcagcggccccgagtttacctttcctctctacggcacgatgggcaacgccgctccacaacaacgcatcgtcgctcagctgggccagggcgtctaccgcaccctgagctccaccctgtaccgcaggcccttcaacatcggtatcaacaaccagcagctgtccgtcctggatggcactgagttcgcctacggcacctcctccaacctgccctccgctgtctaccgcaagagcggcacggtggattccctggacgagatcccaccacagaacaacaatgtgccccccaggcagggtttttcccacaggctcagccacgtgtccatgttccgctccggcttcagcaactcgtccgtgagcatcatcagagctcctatgttctcctggattcatcgcagcgcggagttcaacaatatcattccgtcctcccaaatcacccaaatccccctcaccaagtccaccaacctgggcagcggcacctccgtggtgaagggcccaggcttcacgggcggcgacatcctgcgcaggacctccccgggccagatcagcaccctccgcgtcaacatcaccgctcccctgtcccagaggtaccgcgtcaggattcgctacgctagcaccaccaacctgcaattccacacctccatcgacggcaggccgatcaatcagggtaacttctccgccaccatgtccagcggcagcaacctccaatccggcagcttccgcaccgtgggtttcaccacccccttcaacttctccaacggctccagcgttttcaccctgagcgcccacgtgttcaattccggcaatgaggtgtacattgaccgcattgagttcgtgccagccgaggtcaccttcgaagccgagtacgacctggagagagcccagaaggctgtcaatgagctcttcacgtccagcaatcagatcggcctgaagaccgacgtcactgactaccacatcgaccaagtctccaacctcgtggagtgcctctccgatgagttctgcctcgacgagaagaaggagctgtccgagaaggtgaagcatgccaagcgtctcagcgacgagaggaatctcctccaggaccccaatttccgcggcatcaacaggcagctcgaccgcggctggcgcggcagcaccgacatcacgatccagggcggcgacgatgtgttcaaggagaactacgtgactctcctgggcactttcgacgagtgctaccctacctacttgtaccagaagatcgatgagtccaagctcaaggcttacactcgctaccagctccgcggctacatcgaagacagccaagacctcgagatttacctgatccgctacaacgccaagcacgagaccgtcaacgtgcccggtactggttccctctggccgctgagcgcccccagcccgatcggcaagtgtgcccaccacagccaccacttctccttggacatcgatgtgggctgcaccgacctgaacgaggactttcggtag

SEQ ID NO: 5 - DNA, 1848 bp, Artificial
  Synthetic oligonucleotide (Cry1Ab-02 nucleotide sequence)
atggacaacaacccaaacatcaacgaatgcattccatacaactgcttgagtaacccagaagttgaagtacttggtggagaacgcattgaaaccggttacactcccatcgacatctccttgtccttgacacagtttctgctcagcgagttcgtgccaggtgctgggttcgttctcggactagttgacatcatctggggtatctttggtccatctcaatgggatgcattcctggtgcaaattgagcagttgatcaaccagaggatcgaagagttcgccaggaaccaggccatctctaggttggaaggattgagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgattggaaactacaccgaccacgctgttcgttggtacaacactggcttggagcgtgtctggggtcctgattctagagattggattagatacaaccagttcaggagagaattgaccctcacagttttggacattgtgtctctcttcccgaactatgactccagaacctaccctatccgtacagtgtcccaacttaccagagaaatctatactaacccagttcttgagaacttcgacggtagcttccgtggttctgcccaaggtatcgaaggctccatcaggagcccacacttgatggacatcttgaacagcataactatctacaccgatgctcacagaggagagtattactggtctggacaccagatcatggcctctccagttggattcagcgggcccgagtttacctttcctctctatggaactatgggaaacgccgctccacaacaacgtatcgttgctcaactaggtcagggtgtctacagaaccttgtcttccaccttgtacagaagacccttcaatatcggtatcaacaaccagcaactttccgttcttgacggaacagagttcgcctatggaacctcttctaacttgccatccgctgtttacagaaagagcggaaccgttgattccttggacgaaatcccaccacagaacaacaatgtgccacccaggcaaggattctcccacaggttgagccacgtgtccatgttccgttccggattcagcaacagttccgtgagcatcatcagagctcctatgttctcatggattcatcgtagtgctgagttcaacaatatcattccttcctctcaaatcacccaaatcccattgaccaagtctactaaccttggatctggaacttctgtcgtgaaaggaccaggcttcacaggaggtgatattcttagaagaacttctcctggccagattagcaccctcagagttaacatcactgcaccactttctcaaagatatcgtgtcaggattcgttacgcatctaccactaacttgcaattccacacctccatcgacggaaggcctatcaatcagggtaacttctccgcaaccatgtcaagcggcagcaacttgcaatccggcagcttcagaaccgtcggtttcactactcctttcaacttctctaacggatcaagcgttttcacccttagcgctcatgtgttcaattctggcaatgaagtgtacattgaccgtattgagtttgtgcctgccgaagttaccttcgaggctgagtactga

SEQ ID NO: 6 - DNA, 2100 bp, Artificial
  Synthetic oligonucleotide (Cry1Ah-01 nucleotide sequence)
atgaagaacagcatcaagctgagcgagctgtggtacttcaacgagaggaagtggaggtacttcatggagatcgtgaacaaccagaaccagtgcgtgccgtacaactgcctgaacaacccggagatcgagatcctggagggcggcaggatcagcgtgggcaacaccccgatcgacatcagcctgagcctgacccagttcctgctgagcgagttcgtgccgggcgccggcttcgtgctgggcctgatcgacctgatctggggcttcgtgggcccgagccagtgggacgccttcctggcccaggtggagcagctgatcaaccagaggatcgccgaggccgtgaggaacaccgccatccaggagctggagggcatggccagggtgtacaggacctacgccaccgccttcgccgagtgggagaaggccccggacgacccggagctgagggaggccctgaggacccagttcaccgccaccgagacctacatcagcggcaggatcagcgtgctgaagatccagaccttcgaggtgcagctgctgagcgtgttcgcccaggccgccaacctgcacctgagcctgctgagggacgtggtgttcttcggccagaggtggggcttcagcaccaccaccgtgaacaactactacaacgacctgaccgagggcatcagcacctacaccgactacgccgtgaggtggtacaacaccggcctggagagggtgtggggcccggacagcagggactgggtgaggtacaaccagttcaggagggagctgaccctgaccgtgctggacatcgtggccctgttcccgaactacgacagcaggcgctacccgatcaggaccgtgagccagctgaccagggagatctacaccaacccggtgctggagaacttcgacggcagcttcaggggcagcgcccagggcatcgagaggagcatcaggagcccgcacctgatggacatcctgaacagcatcaccatctacaccgacgcccacaggggctactactactggagcggccaccagatcatggccagcccggtgggcttcagcggcccggagttcaccttcccgctgtacggcaccatgggcaacgccgccccgcagcagaggatcgtggcccagctgggccagggcgtgtacaggaccctgagcagcaccttctacaggaggccgttcaacatcggcatcaacaaccagcagctgagcgtgctggacggcaccgagttcgcctacggcaccagcagcaacctgccgagcgccgtgtacaggaagagcggcaccgtggacagcctggacgagatcccgccgcagaacaacaacgtgccgccgaggcagggcttcagccacaggctgagccacgtgagcatgttcaggagcggcagcagcagcagcgtgagcatcatcagggccccgatgttcagctggattcacaggagcgccgagttcaacaacatcatcgccagcgacagcatcacccagatcccggccgtgaagggcaacttcctgttcaacggcagcgtgatcagcggcccgggcttcaccggcggcgacctggtgaggctgaacagcagcggcaacaacatccagaacaggggctacatcgaggtgccgatccacttcccgagcaccagcaccaggtacagggtgagggtgaggtacgccagcgtgaccccgatccacctgaacgtgaactggggcaacagcagcatcttcagcaacaccgtgccggccaccgccaccagcctggacaacctgcagagcagcgacttcggctacttcgagagcgccaacgccttcaccagcagcctgggcaacatcgtgggcgtgaggaacttcagcggcaccgccggcgtgatcatcgacaggttcgagttcatcccggtgaccgccaccctggaggccgagtacaacctggagcgcgcccagaaggccgtgaacgccctgttcaccagcagcaaccagatcggcctgaagaccgacgtgaccgactaccacatcgaccaggtgagcaacctggtggagtgcctgagcgacgagttctgcctggacgagaagaaggagctgagcgagaaggtgaagcacgccaagaggctgagctga

SEQ ID NO: 7 - DNA, 2100 bp, Artificial
  Synthetic oligonucleotide (Cry1Ah-02 nucleotide sequence)
atgaagaatagcatcaagctgtcggagctgtggtacttcaacgagcgcaagtggaggtacttcatggagatcgtcaacaatcagaatcagtgcgtcccatacaactgcctcaacaatcctgagatcgagattctggagggcgggaggatctccgtgggcaacactccgatcgacatttctctctcactgacccagttcctcctgagcgagttcgtgccaggcgctgggttcgtcctcggcctgatcgacctcatttggggcttcgtcgggccatcccagtgggatgcgttcctcgctcaggttgagcagctgatcaatcagcgcatcgccgaggctgtgaggaacaccgccatccaggagctggagggcatggctagggtctaccggacctacgctacggctttcgctgagtgggagaaggccccggacgatccagagctcagggaggctctgaggacccagttcactgccaccgagacgtacatctccggcaggattagcgtgctcaagatccagacgttcgaggtccagctcctgtctgttttcgcgcaggccgcgaacctccacctgtcactcctgcgcgacgtggtcttcttcggccagcgctgggggttcagcaccacgacagtcaacaattactacaacgacctcactgagggcatctcgacatacactgattacgccgtgcggtggtacaacaccgggctggagagggtttggggcccagacagcagggattgggtgcggtacaatcagttccgcagggagctcaccctgacggtgctcgacatcgtcgcgctgttcccgaactacgattcacggcgctaccccatcaggaccgtctcccagctcacgcgggagatctacacaaatccagttctggagaacttcgacggctcgttccgcgggtctgctcagggcatcgagaggtcaattaggtcccctcatctcatggacatcctgaactcgatcacaatctacactgatgcgcacagggggtactactactggtctggccatcagatcatggcttcaccagtgggcttctccgggccagagttcactttccccctgtacggcaccatgggcaacgccgccccgcagcagcgcatcgttgctcagctcggccagggggtgtacaggacactgtccagcactttctacaggcggccgttcaacatcggcattaacaatcagcagctctccgtcctggacgggacggagttcgcttacggcacatcgtctaacctcccaagcgctgtctaccgcaagagcggcacggttgactcgctggatgagatcccgccccagaacaataacgtgccacctcggcagggcttctcccacaggctcagccatgtttcgatgttcaggtccggctcatccagctcggtgagcatcattagggcccccatgttctcttggatccaccggtcagcggagttcaataacatcatcgcctccgacagcatcacccagattccagcggtcaaggggaatttcctgttcaacggctctgttatctcaggccctgggttcaccggcggggatctcgtccggctgaattcttcagggaataacattcagaaccgcggctacatcgaggtgccaattcacttcccttcgacatctactcgctacagggttcgggtgcgctacgctagcgtgacgccaatccacctcaacgtgaactggggcaattccagcattttcagcaacacagtgcctgccaccgcgacgtcgctcgacaatctgcagtcgtctgatttcggctacttcgagtccgctaacgccttcacgtcatccctggggaatatcgtcggcgttaggaacttcagcggcacagcgggcgtgatcatcgaccggttcgagttcatcccggtcacagccactctcgaggcggagtacaacctggagagggctcagaaggctgtgaacgccctcttcacctcctcgaaccagatcggcctgaagaccgacgtgacggattaccacatcgaccaggtttcgaacctcgtggagtgcctgtctgatgagttctgcctggatgagaagaaggagctgtctgagaaggtgaagcacgccaagcggctgtcgtag

SEQ ID NO: 8 - DNA, 1818 bp, Artificial
  Synthetic oligonucleotide (Cry1Fa nucleotide sequence)
atggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaa

SEQ ID NO: 9 - DNA, 1947 bp, Artificial
  Synthetic oligonucleotide (Cry1Ie nucleotide sequence)
atgaagctcaagaacccagacaagcaccaaagcctgtctagtaacgctaaagtggacaagattgctactgacagtctcaagaacgaaacagacattgagttgaagaacatcaaccacgaagactttcttaggatgtcagagcatgagagcattgatcctttcgtcagtgcctccacaatccaaacgggcatcggaattgctggtaagatcctcggtactttgggtgttcctttcgctggacagattgccagtctctacagtttcatcttgggcgaactctggcctaagggcaagagccaatgggaaatcttcatggaacatgtcgaagagcttatcgaccaaaagatcagtacttacgccaggaacattgcccttgcagacttgaaaggcttgggcgatgccttggctgtctaccacgaaagcttggagagttggatcaagaaccgcaacaacgcaagggctacaagcgttgtcaagagccagtacatcgccttggaactcctctttgttcaaaagctgccatcattcgctgtgtcaggtgaggaagttccactcttgcctatctatgctcaagctgcaaacctccacttactcctcctaagagacgcttctgtgttcggaaaagagtggggactctctaactcgcaaatcagcacattctacaaccgtcaagtggaaagaactagtgactactccgaccactgcgtgaagtggtacagcactggactcaacaacttgagaggtacaaatgccgaaagctgggtccgttacaatcagtttcgtaaggatatgactctcatggtgctcgatctcattgcactcttcccaagctatgacacactcgtgtatcctatcaagaccacttctcaactcacaagagaagtgtataccgacgcaatcgggacagttcatccaaacgccagcttcgcatcaacaacctggtataacaacaatgcaccttccttctctgccattgagtcagctgttgttcgtaatccacatctactcgacttcttggaacaagttacaatctacagcttgctctctagatggagtaacactcagtacatgaacatgtggggaggacacagactggagtttcgtacaatcggtggagttctcaacacttcaacacaaggatctactaacacttccatcaatcctgtgacattgccattcacctctcgtgacgtctatcgtactgaatcattggcaggactgaatctcttcctcactcaacctgttaacggagttccaagggttgacttccattggaagttcgccacacttccaatcgcctctgataacttctactatctcggatatgctggagttggtactcaactccaagactcagagaatgaactcccacctgaaaccactggacagccaaactatgaatcctattcccatagattgtcccacatcggactcattagtgcatcccacgtgaaggcattggtgtactcttggacccatcgtagcgcagatcgtactaacaccattgagcctaacagcatcacacaaatcccattggtgaaggccttcaatctctctagtggtgccgctgttgttagaggaccaggattcacaggtggagacattcttcgtaggactaacactggtacattcggagatatccgtgtgaacatcaacccaccatttgcacaacgttatcgcgttaggattcgctatgcctccactacagatctccaattccacacctccatcaatggtaaagctatcaatcagggtaacttctcagcaaccatgaacagaggagaggacctcgactacaagacctttaggactgtgggctttaccactccattcagcttctccgacgtgcaaagtaccttcaccattggtgcttggaacttctcttccggtaacgaagtttacatcgatcgtattgagtttgtgccagttgaagttacctacgagtga

SEQ ID NO: 10 - DNA, 1992 bp, Artificial
  Synthetic oligonucleotide (Promoter of Maize Ubiquitin gene)
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 11 - DNA, 253 bp, Artificial
  Synthetic oligonucleotide (Terminator of nopaline synthase gene)
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 12 - DNA, 1176 bp, Artificial
  Synthetic oligonucleotide (Phosphomannose isomerase gene)
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 13 - DNA, 19 bp, Artificial Sequence
  Synthetic oligonucleotide (Primer 1)
cgaactacgactcccgcac

SEQ ID NO: 14 - DNA, 21 bp, Artificial Sequence
  Synthetic oligonucleotide (Primer 2)
gtagatttcgcgggtcagttg

SEQ ID NO: 15 - DNA, 22 bp, Artificial Sequence
  Synthetic oligonucleotide (Probe 1)
ctacccgatccgcaccgtgtcc

SEQ ID NO: 16 - DNA, 23 bp, Artificial Sequence
  Synthetic oligonucleotide (Primer 3)
tgcgtattcaattcaacgacatg

SEQ ID NO: 17 - DNA, 23 bp, Artificial Sequence
  Synthetic oligonucleotide (Primer 4)
cttggtagttctggactgcgaac

SEQ ID NO: 18 - DNA, 24 bp, Artificial Sequence
  Synthetic oligonucleotide (Probe 2)
cagcgccttgaccacagctatccc

SEQ ID NO: 19 - DNA, 27 bp, Artificial Sequence
  Synthetic oligonucleotide (Primer 5)
cagtcaggaactacagttgtaagaggg

SEQ ID NO: 20 - DNA, 21 bp, Artificial Sequence
  Synthetic oligonucleotide (Primer 6)
acgcgaatggtcctccactag

SEQ ID NO: 21 - DNA, 27 bp, Artificial Sequence
  Synthetic oligonucleotide (Probe 3)
cgtcgaagaatgtctcctcccgtgaac

SEQ ID NO: 22 - DNA, 23 bp, Artificial Sequence
  Synthetic oligonucleotide (Primer 7)
atcgtgaacaaccagaaccagtg

SEQ ID NO: 23 - DNA, 20 bp, Artificial Sequence
  Synthetic oligonucleotide (Primer 8)
ctccaggatctcgatctccg

SEQ ID NO: 24 - DNA, 26 bp, Artificial Sequence
  Synthetic oligonucleotide (Probe 4)
cgtgccgtacaactgcctgaacaacc

SEQ ID NO: 25 - DNA, 18 bp, Artificial Sequence
  Synthetic oligonucleotide (Primer 9)
tcatttggggcttcgtcg

SEQ ID NO: 26 - DNA, 22 bp, Artificial Sequence
  Synthetic oligonucleotide (Primer 10)
tgattgatcagctgctcaacct

SEQ ID NO: 27 - DNA, 23 bp, Artificial Sequence
  Synthetic oligonucleotide (Probe 5)
ccagtgggatgcgttcctcgctc

SEQ ID NO: 28 - DNA, 23 bp, Artificial Sequence
  Synthetic oligonucleotide (Primer 11)
gagcattgatcctttcgtcagtg

SEQ ID NO: 29 - DNA, 24 bp, Artificial Sequence
  Synthetic oligonucleotide (Primer 12)
caaagtaccgaggatcttaccagc

SEQ ID NO: 30 - DNA, 24 bp, Artificial Sequence
  Synthetic oligonucleotide (Probe 6)
cctccacaatccaaacgggcatcg